(19) United States
(12) Patent Application Publication
(10) Pub. No.: US 2025/0064804 A1
(43) Pub. Date: Feb. 27, 2025

(54) COMBINATION THERAPY FOR TREATING ABNORMAL CELL GROWTH

(71) Applicants: Verastem, Inc., Needham, MA (US); THE UNIVERSITY OF NORTH CAROLINA AT CHAPEL HILL, Chapel Hill, NC (US)

(72) Inventors: SANJIB CHOWDHURY, Needham, MA (US); JONATHAN A. PACHTER, Needham, MA (US); SILVIA COMA, Needham, MA (US); KIRSTEN LEIGH BRYANT, Chapel Hill, NC (US); CHANNING JOSEPH DER, Chapel Hill, NC (US); WEN-HSUAN CHANG, Chapel Hill, NC (US); JONATHAN MICHAEL DELIBERTY, Chapel Hill, NC (US)

(21) Appl. No.: 18/718,137

(22) PCT Filed: Dec. 9, 2022
(86) PCT No.: PCT/US2022/081248

§ 371 Date: Jun. 10, 2024

Related U.S. Application Data
(60) Provisional application No. 63/265,240, filed on Dec. 10, 2021.

(51) Int. Cl. A61K 31/506; A61K 31/4706; A61K 31/5377; A61P 35/00

(52) U.S. Cl. CPC A61K 31/506; A61K 31/4706; A61K 31/5377; A61P 35/00

(57) ABSTRACT

The present invention relates, in part, to methods, compounds, and compositions of an autophagy pathway inhibitor in combination with a dual RAF/MEK inhibitor, for treating abnormal cell growth (e.g., cancer).

### CROSS REFERENCE TO RELATED APPLICATIONS

This application claims the benefit of and priority to U.S. Provisional Patent Application No. 63/265,240 filed Dec. 10, 2021, the entire contents of which are incorporated herein by reference.

### STATEMENT AS TO FEDERALLY SPONSORED RESEARCH

This invention was made with government support under Grant Number CA232113 awarded by the National Institutes of Health. The United States government has certain rights in the invention.

### BACKGROUND

Components of the RAS/RAF/MEK/ERK (MAPK) signal transduction pathway represent opportunities for the treatment of abnormal cell growth, e.g., cancer. Mutations in RAS/RAF/MEK/ERK are frequently found in human cancers. These mutants result in a constitutively active MAPK kinase cascade, leading to tumor cell proliferation, differentiation, survival, and migration. Selective inhibitors of certain components of the RAS/RAF/MEK/ERK signal transduction pathway, such as RAS, RAF, MEK and ERK, are useful in the treatment of abnormal cell growth, in particular cancer, in mammals.

Due to the severity and breadth of diseases and disorders associated with abnormal cell growth, e.g., cancer, there is a need for effective therapeutic means and methods for treatment. The compounds, compositions, and methods described herein are directed toward this end.

### SUMMARY

The present disclosure provides, in part, combinations (e.g., combinations of compounds as described herein, e.g., a dual RAF/MEK inhibitor and an autophagy pathway inhibitor), which can be used, for example, in methods of treating abnormal cell growth (e.g., cancer) in a subject in need thereof.

Thus, in an aspect, provided herein is a method of treating a cancer in a subject in need thereof, the method comprising administering to the subject an effective amount of a dual RAF/MEK inhibitor and an effective amount of an autophagy pathway inhibitor.

In some embodiments, the dual RAF/MEK inhibitor is a compound of formula (I):

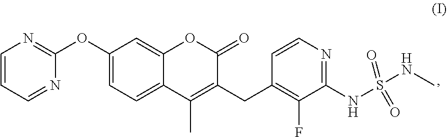 [SMILES: CNS(=O)(=O)Nc1nccc(Cc2c(C)c3ccc(Oc4ncccn4)cc3oc2=O)c1F]

or a pharmaceutically acceptable salt thereof.

In some embodiments, the dual RAF/MEK inhibitor is a compound of formula (II):

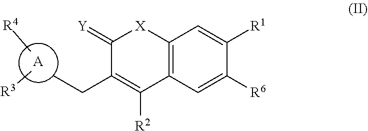

including pharmaceutically acceptable salts thereof, wherein the variables are as defined herein.

In some embodiments, the dual RAF/MEK inhibitor is IMM-1-104, or a pharmaceutically acceptable salt thereof.

In some embodiments, the dual RAF/MEK inhibitor is IK-595, or a pharmaceutically acceptable salt thereof.

Other objects and advantages will become apparent to those skilled in the art from a consideration of the ensuing Detailed Description, Examples, and Claims.

### BRIEF DESCRIPTION OF THE DRAWINGS

FIG. 1 illustrates autophagic flux effects of VS-6766 in exemplary KRAS mutant pancreatic cell lines.

FIG. 2 illustrates synergistic effects of VS-6766 and autophagy pathway inhibitors in exemplary KRAS mutant pancreatic cell lines.

### DETAILED DESCRIPTION

As generally described herein, the present disclosure provides, in part, methods useful for treating abnormal cell growth (e.g., cancer) in a subject in need thereof, comprising administering to the subject an effective amount of a dual RAF/MEK inhibitor and an effective amount of an autophagy pathway inhibitor.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In some embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In some embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In some embodiments, the enantiomerically pure S-compound in such compositions can, for example, comprise, at least about 95% by weight S-compound and at most about 5% by weight R-compound, by total weight of the compound. In some embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below.

The term “halogen atom,” as used herein, means any one of the radio stable atoms of column 7 of the Periodic Table of the Elements, e.g., fluorine, chlorine, bromine, or iodine, with fluorine and chlorine being preferred.

The term “ester,” as used herein, refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

The term “amide,” as used herein, refers to a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxyl, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, NY, 1999, which is incorporated herein in its entirety.

The term “aromatic,” as used herein, refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “hetero aromatic” refers to an aromatic group which contains at least one heterocyclic ring.

As used herein, “Ca to Cb” in which “a” and “b” are integers refer to the number of carbon atoms in an alkyl, alkenyl or alkynyl group, or the number of carbon atoms in the ring of a cycloalkyl, aryl, heteroaryl or heterocyclyl group. That is, the alkyl, alkenyl, alkynyl, ring of the cycloalkyl, ring of the aryl, ring of the heteroaryl or ring of the heterocyclyl can contain from “a” to “b”, inclusive, carbon atoms. Thus, for example, a “C1 to C4 alkyl” group or a “C1-C4 alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. Likewise, for example, cycloalkyl group may contain from “a” to “b”, inclusive, total atoms, such as a C3-C8 cycloalkyl group, 3 to 8 carbon atoms in the ring(s). If no “a” and “b” are designated with regard to an alkyl, cycloalkyl, or cycloalkenyl, the broadest range described in these definitions is to be assumed. Similarly, a “4 to 7 membered heterocyclyl” group refers to all heterocyclyl groups with 4 to 7 total ring atoms, for example, azetidine, oxetane, oxazoline, pyrrolidine, piperidine, piperazine, morpholine, and the like. As used herein, the term “C1-C6” includes C1, C2, C3, C4, C5 and C6, and a range defined by any of the two preceding numbers. For example, C1-C6 alkyl includes C1, C2, C3, C4, C5 and C6 alkyl, C2-C6 alkyl, C1-C3 alkyl, etc. Similarly, C3-C8 carbocyclyl or cycloalkyl each includes hydrocarbon ring containing 3, 4, 5, 6, 7 and 8 carbon atoms, or a range defined by any of the two numbers, such as C3-C7 cycloalkyl or C5-C6 cycloalkyl. As another example, 3 to 10 membered heterocyclyl includes 3, 4, 5, 6, 7, 8, 9, or 10 ring atoms, or a range defined by any of the two preceding numbers, such as 4 to 6 membered or 5 to 7 membered heterocyclyl.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C1-C4 alkyl” or similar designations. By way of example only, “C1-C4 alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Exemplary alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substituent may be substituted with one of the above substituents.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group of the compounds may be designated as “C2-C4 alkenyl” or similar designations. By way of example only, “C2-C4 alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Exemplary alkenyl groups include, but are in no way limited to, ethenyl, propenyl, butenyl, pentenyl, and hexenyl, and the like.

As used herein, “alkynyl” refers to an alkyl group that contains in the straight or branched hydrocarbon chain one or more triple bonds. An alkynyl group may be unsubstituted or substituted. When substituted, the substituent(s) may be selected from the same groups disclosed above with regard to alkyl group substitution. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group of the compounds may be designated as “C2-C4 alkynyl” or similar designations. By way of example only, “C2-C4 alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Exemplary alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group of the compounds may be designated as “C1-C4 heteroalkyl” or similar designations. The heteroalkyl group may contain one or more heteroatoms. By way of example only, “C1-C4 heteroalkyl” indicates that there are one to four carbon atoms in the heteroalkyl chain and additionally one or more heteroatoms in the backbone of the chain.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on an aryl group may form a non-aromatic ring fused to the aryl group, including a cycloalkyl, cycloalkenyl, cycloalkynyl, and heterocyclyl.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on a heteroayl group may form a non-aromatic ring fused to the aryl group, including a cycloalkyl, cycloalkenyl, cycloalkynyl, and heterocyclyl.

As used herein, an “aralkyl” or “arylalkyl” refers to an aryl group connected, as a substituent, via an alkylene group. The alkylene and aryl group of an aralkyl may be substituted or unsubstituted. Examples include but are not limited to benzyl, substituted benzyl, 2-phenylethyl, 3-phenylpropyl, and naphtylalkyl. In some cases, the alkylene group is a lower alkylene group.

As used herein, a “heteroaralkyl” or “heteroarylalkyl” is heteroaryl group connected, as a substituent, via an alkylene group. The alkylene and heteroaryl group of heteroaralkyl may be substituted or unsubstituted. Examples include but are not limited to 2-thienylmethyl, 3-thienylmethyl, furylmethyl, thienylethyl, pyrrolylalkyl, pyridylalkyl, isoxazollylalkyl, and imidazolylalkyl, and their substituted as well as benzo-fused analogs. In some cases, the alkylene group is a lower alkylene group.

As used herein, a “alkylene” refers to a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C1-C4 alkylene” or similar designations. By way of example only, “C1-C4 alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2, 2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” refers to a straight or branched chain di radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C2-C4 alkenylene” or similar designations. By way of example only, “C2 alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2, 2-dimethyl-ethen-1,1-diyl.

As used herein, “alkylidene” refers to a divalent group, such as =CR′R″, which is attached to one carbon of another group, forming a double bond, alkylidene groups include, but are not limited to, methylidene (=CH₂) and ethylidene (=CHCH₃). As used herein, “arylalkylidene” refers to an alkylidene group in which either R′ and R″ is an aryl group. An alkylidene group may be substituted or unsubstituted.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, amoxy, tert-amoxy and the like. An alkoxy may be substituted or unsubstituted.

As used herein, “alkylthio” refers to the formula —SR wherein R is an alkyl is defined as above, e.g. methylmercapto, ethylmercapto, n-propylmercapto, 1-methylethylmercapto (isopropylmercapto), n-butylmercapto, iso-butylmercapto, sec-butylmercapto, tert-butylmercapto, and the like. An alkylthio may be substituted or unsubstituted.

As used herein, “aryloxy” and “arylthio” refers to RO— and RS—, respectively, in which R is an aryl, such as but not limited to phenyl. Both an aryloxyl and arylthio may be substituted or unsubstituted.

As used herein, “acyl” refers to —C(═O)R, wherein R is hydrogen, C1-C6 alkyl, C2-C6 alkenyl, C2-C6 alkynyl, C3-C8 carbocyclyl, aryl, 5-10 membered heteroaryl, and 5-10 membered heterocyclyl, as defined herein. Non-limiting examples include formyl, acetyl, propanoyl, benzoyl, and acryl.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups may range from C3 to C10, in other embodiments it may range from C3 to C6. A cycloalkyl group may be unsubstituted or substituted. Exemplary cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. When substituted, substituents on a cycloalkyl group may form an aromatic ring fused to the cycloalkyl group, including an aryl and a heteroaryl.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). When composed of two or more rings, the rings may be connected together in a fused, bridged or spiro-connected fashion. A cycloalkenyl group may be unsubstituted or substituted. When substituted, the substituent(s) may be an alkyl or selected from the groups disclosed above with regard to alkyl group substitution unless otherwise indicated. When substituted, substituents on a cycloalkenyl group may form an aromatic ring fused to the cycloalkenyl group, including an aryl and a heteroaryl.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group may be unsubstituted or substituted. When substituted, the substituent(s) may be an alkyl or selected from the groups disclosed above with regard to alkyl group substitution unless otherwise indicated. When substituted, substituents on a cycloalkynyl group may form an aromatic ring fused to the cycloalkynyl group, including an aryl and a heteroaryl.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. The “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl A-oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone. When substituted, substituents on a heteroalicyclyl group may form an aromatic ring fused to the heteroalicyclyl group, including an aryl and a heteroaryl.

As used herein, the term “(cycloalkenyl)alkyl” refers to a cycloalkenyl group connected, as a substituent, via an alkylene group. The alkylene and cycloalkenyl of a (cycloalkenyl)alkyl may be substituted or unsubstituted. In some cases, the alkylene group is a lower alkylene group.

As used herein, the term “(cycloalkynyl)alkyl” to a cycloalkynyl group connected, as a substituent, via an alkylene group. The alkylene and cycloalkynyl of a (cycloalkynyl)alkyl may be substituted or unsubstituted. In some cases, the alkylene group is a lower alkylene group.

As used herein, the term “O-carboxy” refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

As used herein, the term “C-carboxy” refers to a “—C(═O)R” group in which R can be the same as defined with respect to O-carboxy. A C-carboxy may be substituted or unsubstituted.

As used herein, the term “trihalomethanesulfonyl” refers to an “X₃CSO₂—” group wherein X is a halogen.

As used herein, the term “cyano” refers to a “—CN” group.

As used herein, the term “cyanato” refers to an “—OCN” group.

As used herein, the term “isocyanato” refers to a “—NCO” group.

As used herein, the term “thiocyanato” refers to a “—SCN” group.

As used herein, the term “isothiocyanato” refers to an “—NCS” group.

As used herein, the term “sulfinyl” refers to a “—S(═O)—R” group in which R can be the same as defined with respect to O-carboxy. A sulfinyl may be substituted or unsubstituted.

As used herein, the term “sulfonyl” refers to an “—SO₂R” group in which R can be the same as defined with respect to O-carboxy. A sulfonyl may be substituted or unsubstituted.

As used herein, the term “S-sulfonamido” refers to a “—SO₂NRARB” group in which RA and RB can be the same as defined with respect to O-carboxy. An S-sulfonamido may be substituted or unsubstituted.

As used herein, the term “N-sulfonamido” refers to a “—SO₂N(RA)(RB)” group in which RA and RB can be the same as defined with respect to O-carboxy. A sulfonyl may be substituted or unsubstituted.

As used herein, the term “trihalomethanesulfonamido” refers to an “X₃CSO₂N(R)—” group with X as halogen and R can be the same as defined with respect to O-carboxy. A trihalomethanesulfonamido may be substituted or unsubstituted.

As used herein, the term “O-carbamyl” refers to a “—OC(═O)NRARB” group in which RA and RB can be the same as defined with respect to O-carboxy. An O-carbamyl may be substituted or unsubstituted.

As used herein, the term “N-carbamyl” refers to an “ROC(═O)NRA group in which R and RA can be the same as defined with respect to O-carboxy. An N-carbamyl may be substituted or unsubstituted.

As used herein, the term “O-thiocarbamyl” refers to a “—OC(═S)—NRARB” group in which RA and RB can be the same as defined with respect to O-carboxy. An O-thiocarbamyl may be substituted or unsubstituted.

As used herein, the term “N-thiocarbamyl” refers to an “ROC(═S)NRA-” group in which R and RA can be the same as defined with respect to O-carboxy. An N-thiocarbamyl may be substituted or unsubstituted.

As used herein, the term “C-amido” refers to a “—C(═O)NRARB” group in which RA and RB can be the same as defined with respect to O-carboxy. A C-amido may be substituted or unsubstituted.

As used herein, the term “N-amido” refers to a “RC(═O)NRA-” group in which R and RA can be the same as defined with respect to O-carboxy. An N-amido may be substituted or unsubstituted.

As used herein, the term “amino” refers to a “—NRARB” group in which RA and RB are each independently selected from hydrogen, C1-C6 alkyl, C2-C6 alkenyl, C2-C6 alkynyl, C3-C8 carbocyclyl, C6-C10 aryl, 5-10 membered heteroaryl, and 5-10 membered heterocyclyl, as defined herein.

As used herein, the term “aminoalkyl” refers to an amino group connected via an alkylene group.

As used herein, the term “ester” refers to a “—C(═O)OR” group in which R can be the same as defined with respect to O-carboxy. An ester may be substituted or unsubstituted.

As used herein, the term “lower aminoalkyl” refers to an amino group connected via a lower alkylene group. A lower aminoalkyl may be substituted or unsubstituted.

As used herein, the term “lower alkoxyalkyl” refers to an alkoxy group connected via a lower alkylene group. A lower alkoxyalkyl may be substituted or unsubstituted.

As used herein, the term “acetyl” refers to a —C(═O)CH₃, group.

As used herein, the term “perhaloalkyl” refers to an alkyl group where all of the hydrogen atoms are replaced by halogen atoms.

As used herein, the term “carbocyclyl” refers to a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C3-C6 carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, the term “(cycloalkyl)alkyl” refers to a cycloalkyl group connected, as a substituent, via an alkylene group. The alkylene and cycloalkyl of a (cycloalkyl)alkyl may be substituted or unsubstituted. Examples include but are not limited cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, the term “cycloalkyl” refers to a fully saturated carbocyclyl ring or ring system. Examples include cyclopropyl, cyclobutyl, cyclopentyl, and cyclohexyl.

As used herein, the term “cycloalkenyl” means a carbocyclyl ring or ring system having at least one double bond, wherein no ring in the ring system is aromatic. An example is cyclohexenyl.

As used herein, the term “heterocyclyl” refers to three-, four-, five-, six-, seven-, and eight- or more membered rings wherein carbon atoms together with from 1 to 3 heteroatoms constitute said ring. A heterocyclyl can optionally contain one or more unsaturated bonds situated in such a way, however, that an aromatic pi-electron system does not arise. The heteroatoms are independently selected from oxygen, sulfur, and nitrogen. A heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like. A “heterocyclyl” can refer to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2//-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, the term “(heterocyclyl)alkyl” refers to a heterocyclyl group connected, as a substituent, via an alkylene group. Examples include, but are not limited to, imidazolinylmethyl and indolinylethyl.

Substituted groups are based upon or derived from the unsubstituted parent group in which there has been an exchange of one or more hydrogen atoms for another atom or group. Unless otherwise indicated, when a group is deemed to be “substituted,” the group is substituted with one or more substituents independently selected from C1-C6 alkyl, C2-C6 alkenyl, C2-C6 alkynyl, C1-C6 heteroalkyl, C3-C7 carbocyclyl (optionally substituted with halo, C1-C6 alkyl, C1-C6 alkoxy, C1-C6 haloalkyl, and C1-C6 haloalkoxy), C3-C7-carbocyclyl-C1-C6-alkyl (optionally substituted with halo, C1-C6 alkyl, C1-C6 alkoxy, C1-C6 haloalkyl, and C1-C6 haloalkoxy), 5-10 membered heterocyclyl (optionally substituted with halo, C1-C6 alkyl, C1-C6 alkoxy, C1-C6 haloalkyl, and C1-C6 haloalkoxy), 5-10 membered heterocyclyl-C1-C6-alkyl (optionally substituted with halo, C1-C6 alkyl, C1-C6 alkoxy, C1-C6 haloalkyl, and C1-C6 haloalkoxy), aryl (optionally substituted with halo, C1-C6 alkyl, C1-C6 alkoxy, C1-C6 haloalkyl, and C1-C6 haloalkoxy), aryl(C1-C6)alkyl (optionally substituted with halo, C1-C6 alkyl, C1-C6 alkoxy, C1-C6 haloalkyl, and C1-C6 haloalkoxy), 5-10 membered heteroaryl (optionally substituted with halo, C1-C6 alkyl, C1-C6 alkoxy, C1-C6 haloalkyl, and C1-C6 haloalkoxy), 5-10 membered heteroaryl(C1-C6)alkyl (optionally substituted with halo, C1-C6 alkyl, C1-C6 alkoxy, C1-C6 haloalkyl, and C1-C6 haloalkoxy), halo, cyano, hydroxy, C1-C6 alkoxy, C1-C6 alkoxy(C1-C6)alkyl (i.e., ether), aryloxy, sulfhydryl (mercapto), halo(C1-C6)alkyl (e.g., —CF₃), halo(C1-C6)alkoxy (e.g., —OCF₃), C1-C6 alkylthio, arylthio, amino, amino(C1-C6)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (=O). Wherever a group is described as “optionally substituted” that group can be substituted with the above substituents.

In some embodiments, a substituted group is substituted with one or more substituent(s) individually and independently selected from C1-C4 alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Unless otherwise indicated, when a substituent is deemed to be “optionally substituted,” it is meant that the substituent” is a group that may be substituted with one or more group(s) individually and independently selected from alkyl, alkenyl, alkynyl, cycloalkyl, aryl, heteroaryl, heteroalicyclic, hydroxyl, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

### Other Definitions

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, “prophylactic treatment” contemplates an action that occurs before a subject begins to suffer from the specified disease, disorder or condition.

The term, “oral dosage form,” as used herein, refers to a composition or medium used to administer an agent to a subject. Typically, an oral dosage form is administered via the mouth, however, “oral dosage form” is intended to cover any substance which is administered to a subject and is absorbed across a membrane, e.g., a mucosal membrane, of the gastrointestinal tract, including, e.g., the mouth, esophagus, stomach, small intestine, large intestine, and colon. For example, “oral dosage form” covers a solution which is administered through a feeding tube into the stomach.

A “cycle”, as used herein in the context of a cycle of administration of a drug, refers to a period of time for which a drug is administered and may further include a rest period of not administering the drug to a subject. In some embodiments, one cycle is four weeks.

A “RAS mutation” is a mutation in the RAS gene. For example, a “KRAS mutation” is a mutation of the KRAS gene (i.e., a nucleic acid mutation) or Kras protein (i.e., an amino acid mutation) that results in aberrant Kras protein function associated with increased and/or constitutive activity by favoring the active GTP-bound state of the Kras protein. The mutation may be at conserved sites that favor GTP binding and constitutively active Kras protein. In some instances, the mutation is at one or more of codons 12, 13, and 16 of the KRAS gene. For example, a KRAS mutation may be at codon 12 of the KRAS gene, for instance, as a single point substitution mutation at codon 12 (i.e., KRAS G12X mutation) (e.g., a KRAS G12V mutation arises from a single nucleotide change (c.35G>T) and results in an amino acid substitution of the glycine (G) at position 12 by a valine (V)). Exemplary KRAS G12X mutations include, but are not limited to, KRAS G12V, KRAS G12D, KRAS G12A, KRAS G12R, KRAS G12S, or KRAS G12C. An “NRAS mutation” is a mutation of the NRAS gene. An “HRAS mutation” is a mutation of the HRAS gene.

A “RAF mutation” is a mutation in the RAF gene. For example, a “BRAF mutation” is a mutation in the BRAF gene. In some instances, the mutation in the BRAF gene is a BRAF V600 mutation including BRAF V600E, BRAF V600K, BRAF V600D, BRAF V600R, and BRAF V600M mutation. An “ARAF mutation” is a mutation in the ARAF gene. A “CRAF mutation” is a mutation in the CRAF gene.

### Methods of Treatment

In an aspect, provided herein is a method of treating a cancer in a subject in need thereof, the method comprising administering to the subject an effective amount of a dual RAF/MEK inhibitor and an effective amount of an autophagy pathway inhibitor.

In some embodiments, the cancer is identified as having one or more KRAS mutation. In some embodiments, the KRAS mutation is a KRAS G12X mutation. In some embodiments, the KRAS mutation is KRAS G12V mutation. In some embodiments, the KRAS mutation is KRAS G12D mutation. In some embodiments, the KRAS mutation is KRAS G12A mutation. In some embodiments, the KRAS mutation is KRAS G12R mutation. In some embodiments, the KRAS mutation is KRAS G12S mutation. In some embodiments, the KRAS mutation is KRAS G12C mutation. In some embodiments, the KRAS mutation is a KRAS G13X mutation. In some embodiments, the KRAS mutation is KRAS G13V mutation. In some embodiments, the KRAS mutation is KRAS G13D mutation. In some embodiments, the KRAS mutation is KRAS G13A mutation. In some embodiments, the KRAS mutation is KRAS G13R mutation. In some embodiments, the KRAS mutation is KRAS G13S mutation. In some embodiments, the KRAS mutation is KRAS G13E mutation. In some embodiments, the KRAS mutation is KRAS G12 dup mutation. In some embodiments, the KRAS mutation is KRAS G13C mutation. In some embodiments, the KRAS mutation is a KRAS Q61X mutation. In some embodiments, the KRAS mutation is KRAS Q61H mutation. In some embodiments, the KRAS mutation is KRAS Q61K mutation. In some embodiments, the KRAS mutation is KRAS Q61L mutation. In some embodiments, the KRAS mutation is KRAS Q61R mutation. In some embodiments, the KRAS mutation is KRAS Q61P mutation. In some embodiments, the KRAS mutation is KRAS Q61E mutation.

In some embodiments, the cancer is identified as having a HRAS mutation.

In some embodiments, the cancer is identified as having a NRAS mutation

In some embodiments, the cancer is identified as having a BRAF mutation. In some embodiments, the BRAF mutation is BRAF V600 mutation. In some embodiments, the BRAF V600 mutation is one or more BRAF V600E, BRAF V600K, BRAF V600D, BRAF V600R, or BRAF V600M mutation. In some embodiments, the BRAF V600 mutation is BRAF V600E mutation. In some embodiments, the BRAF V600 mutation is BRAF V600K mutation. In some embodiments, the BRAF V600 mutation is BRAF V600D mutation. In some embodiments, the BRAF V600 mutation is BRAF V600R mutation. In some embodiments, the BRAF V600 mutation is BRAF V600M mutation.

In some embodiments, the cancer is identified as having a ARAF mutation.

In some embodiments, the cancer is identified as having a CRAF mutation.

In some embodiments, the cancer is identified as having MEK1 and/or MEK2 mutation.

In some embodiments, the cancer is identified as having NF1 alterations, KRAS amplification, NRAS amplification, and/or MYC amplification.

In some embodiments, the cancer is identified as having positive phospho-ERK protein expression (e.g., ≥10%, ≥20% or ≥30% of cells) detected by immunohistochemistry.

In some embodiments, the dual RAF/MEK inhibitor is a compound of formula (I):

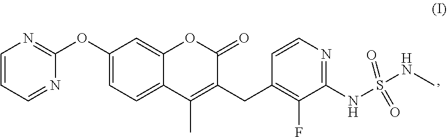 [SMILES: CNS(=O)(=O)Nc1nccc(Cc2c(C)c3ccc(Oc4ncccn4)cc3oc2=O)c1F]

or a pharmaceutically acceptable salt thereof.

In some embodiments, the dual RAF/MEK inhibitor is a compound of formula (I):

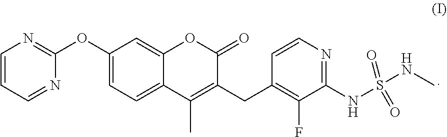 [SMILES: CNS(=O)(=O)Nc1nccc(Cc2c(C)c3ccc(Oc4ncccn4)cc3oc2=O)c1F]

In some embodiments, the dual RAF/MEK inhibitor is a potassium salt of the compound of formula (I).

In some embodiments, the dual RAF/MEK inhibitor is IMM-1-104, or a pharmaceutically acceptable salt thereof.

In some embodiments, the dual RAF/MEK inhibitor is IK-595, or a pharmaceutically acceptable salt thereof.

In some embodiments, the dual RAF/MEK inhibitor is a compound of formula (II):

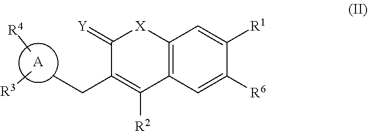

including pharmaceutically acceptable salts thereof, wherein the variables are as defined herein.

In some embodiments, the dual RAF/MEK inhibitor is a compound selected from the compound of Table I, or a pharmaceutically acceptable salt thereof.

In some embodiments, the autophagy pathway inhibitor is AMPK inhibitor, L-type Ca2+ channel activator, USP10 and USP13 inhibitor, VPS34 inhibitor, or ULK inhibitor.

In some embodiments, the autophagy pathway inhibitor is chloroquine, hydroxychloroquine, apilimod, 3-methyladenine, Wortmannin, LY294002, cycloheximide, bafilomycin A1, Lys05, leupeptin, E64d, Pepstatin A, (±)-Bay K 8644, Spautin 1, MRT 67307, MRT 68921, dorsomorphin, rapalogs, NSC-185058, DQ661, DCC-3116, PHY34, ENV-201, ERAS-5, LCC03 (5-(2′,4′-Difluorophenyl)-salicylanilide derivative), PTU (1-phenyl 2-thiourea), siroquine (JP001), SB02024, GCT-VPS34, Vescor01, S-130, SBI-0206965, SMER-28, or FMK-9A.

In some embodiments, the autophagy pathway inhibitor is orally administered to the subject. In some embodiments, the autophagy pathway inhibitor is administered once daily. In some embodiments, the autophagy pathway inhibitor is administered twice daily. In some embodiments, the autophagy pathway inhibitor is dosed at 10 mg to 2000 mg per administration. In some embodiments, the autophagy pathway inhibitor is dosed at 100 mg to 1000 mg per administration.

In some embodiments, the method uses inhibitors of autophagy initiation, nucleation, maturation, or degradation.

In some embodiments, the method uses inhibitors of ULK1, ULK2, ATG13, FIP200, ATG101, VPS34, VPS15, ATG14L, Beclin-1, ATG4B, or lysosome function.

In some embodiments, the dual RAF/MEK inhibitor and the autophagy pathway inhibitor are independently dosed cyclically.

In some embodiments, the dual RAF/MEK inhibitor and the autophagy pathway inhibitor are simultaneously dosed cyclically.

In some embodiments, the methods described herein further comprises administering to the subject an effective amount of a FAK inhibitor (e.g., defactinib, or a pharmaceutically acceptable salt thereof). In some embodiments, the FAK inhibitor (e.g., defactinib, or a pharmaceutically acceptable salt thereof) is dosed at about 100 mg to about 1000 mg. In some embodiments, the FAK inhibitor (e.g., defactinib, or a pharmaceutically acceptable salt thereof) is dosed at about 100 mg to about 400 mg per administration. In some embodiments, the FAK inhibitor (e.g., defactinib, or a pharmaceutically acceptable salt thereof) is dosed at 200 mg per administration. In some embodiments, the FAK inhibitor (e.g., defactinib, or a pharmaceutically acceptable salt thereof) is dosed at 400 mg per administration. In some embodiments, the FAK inhibitor (e.g., defactinib, or a pharmaceutically acceptable salt thereof) is administered twice daily. In some embodiments, the FAK inhibitor (e.g., defactinib, or a pharmaceutically acceptable salt thereof) is dosed as a cycle, comprising administering the FAK inhibitor (e.g., defactinib, or a pharmaceutically acceptable salt thereof) for three weeks and then not administering the FAK inhibitor (e.g., defactinib, or a pharmaceutically acceptable salt thereof) for one week. In some embodiments, the FAK inhibitor (e.g., defactinib, or a pharmaceutically acceptable salt thereof) is administered orally to the subject.

In some embodiments, the dual RAF/MEK inhibitor and the FAK inhibitor (e.g., defactinib, or a pharmaceutically acceptable salt thereof) are independently dosed cyclically.

In some embodiments, the dual RAF/MEK inhibitor and the FAK inhibitor (e.g., defactinib, or a pharmaceutically acceptable salt thereof) are simultaneously dosed cyclically.

In some embodiments, the cancer is pancreatic cancer, gynecologic cancer (e.g., cervical cancer, ovarian cancer, uterine cancer, vaginal cancer, endometrial cancer, or vulvar cancer), liver cancer, prostate cancer, mesothelioma, breast cancer, bladder cancer, melanoma, lung cancer, colorectal cancer, thyroid cancer, glioblastoma, or renal cancer. In some embodiments, the cancer is melanoma, lung cancer, colorectal cancer, thyroid cancer, glioblastoma, or renal cancer. In some embodiments, the lung cancer is non-small cell lung cancer. In some embodiments, the lung cancer is metastatic non-small cell lung cancer. In some embodiments, the melanoma is unresectable melanoma. In some embodiments, the melanoma is metastatic melanoma. In some embodiments, the cancer is colorectal cancer. In some embodiments, the thyroid cancer is papillary thyroid cancer. In some embodiments, the thyroid cancer is follicular thyroid cancer. In some embodiments, the thyroid cancer is anaplastic thyroid cancer.

In an aspect, provided herein is a method of treating a cancer in a subject in need thereof, the method comprising administering to the subject an effective amount of defactinib or a pharmaceutically acceptable salt thereof and an effective amount of an autophagy pathway inhibitor.

### Dual RAF/MEK Inhibitors

An exemplary dual RAF/MEK inhibitor described herein is VS-6766 (also referred to as CKI27, CH5126766, RO5126766, or avutometinib).

In some embodiments, the dual RAF/MEK inhibitor is a compound of formula (I):

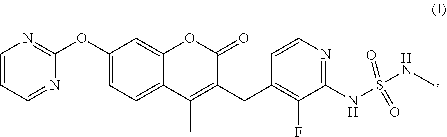 [SMILES: CNS(=O)(=O)Nc1nccc(Cc2c(C)c3ccc(Oc4ncccn4)cc3oc2=O)c1F]

or a pharmaceutically acceptable salt thereof.

In some embodiments, the compound of formula (I) is:

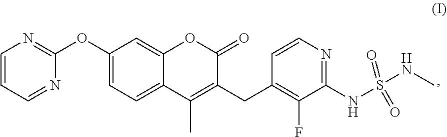 [SMILES: CNS(=O)(=O)Nc1nccc(Cc2c(C)c3ccc(Oc4ncccn4)cc3oc2=O)c1F]

which is also referred to herein as Compound 1 or VS-6766 free form.

In some embodiments, the dual RAF/MEK inhibitor is a pharmaceutically acceptable salt of the compound of formula (I). In some embodiments, the dual RAF/MEK inhibitor is a potassium salt of the compound of formula (I), which is also referred to as VS-6766. Other pharmaceutically acceptable salts of the compound of formula (I) are contemplated herein.

In some embodiments, the dual RAF/MEK inhibitor is a compound having the structure of Formula (II):

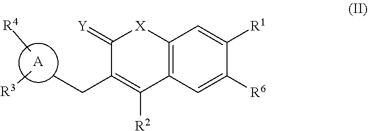

including pharmaceutically acceptable salts thereof, wherein:
Ring A is

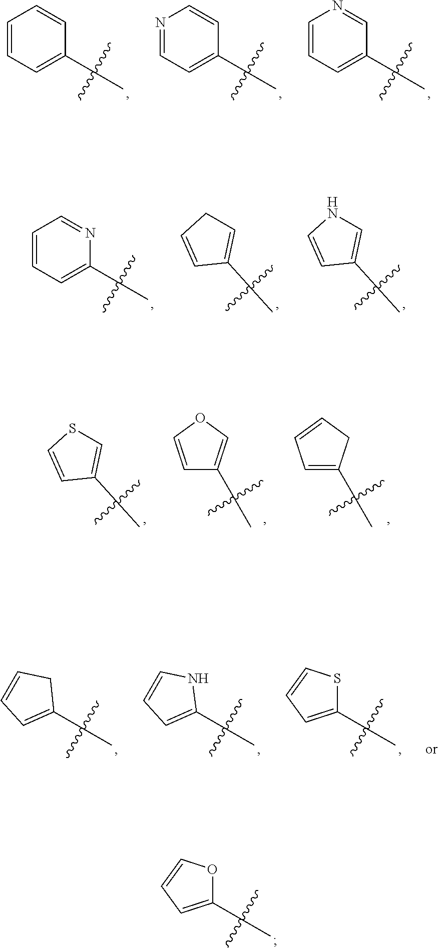

R¹, R², R³, and R⁴ are each independently selected from the group consisting of H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C1 to C6 alkoxy, optionally substituted C1 to C6 alkyl, optionally substituted C2 to C6 alkenyl, optionally substituted C2 to C6 alkynyl, optionally substituted C3 to C8 cycloalkyl, optionally substituted C6 to C10 aryl, optionally substituted C3 to C8 heterocyclyl, optionally substituted C3 to C10 heteroaryl, and L; R⁶ is selected from the group consisting of H, deuterium, hydroxyl, halogen, cyano, nitro, optionally substituted amino, optionally substituted C1 to C6 alkoxy, optionally substituted C1 to C6 alkyl, optionally substituted C2 to C6 alkenyl, and optionally substituted C2 to C6 alkynyl;
X is C(R⁵)₂, CH(R⁵), CH₂, —O—,

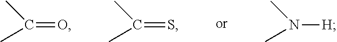

L is —Z₁—Z₂ or —Z₁—Z₂—Z₃;
Z₁, Z₂, and Z₃ are independently selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C1 to C6 alkyl, optionally substituted C3 to C8 cycloalkyl, optionally substituted C6 to C10 aryl, optionally substituted C3 to C8 heterocyclyl, optionally substituted C3 to C10 heteroaryl, —CH₂— (optionally substituted aryl), —CH₂— (optionally substituted C3 to C8 cycloalkyl), and —CH₂— (optionally substituted C3 to C10 heteroaryl); each R⁵ and R⁵ are independently selected from H, deuterium, optionally substituted C1 to C6 alkyl, optionally substituted C2 to C6 alkenyl, optionally substituted C2 to C6 alkynyl, optionally substituted C3 to C8 carbocyclyl, optionally substituted C6 to C10 aryl, optionally substituted C3 to C8 heterocyclyl, and optionally substituted C3 to C10 heteroaryl; and
Y is CH₂, NH, or O, with the proviso that R¹ is not —O-pyrimidyl.

In some embodiments, the dual RAF/MEK inhibitor is a compound selected from a compound in Table I:

TABLE I

| No. | Structure |
|---|---|
| 7 | 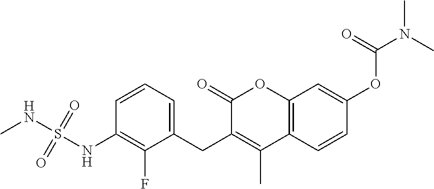 |
| 9 | 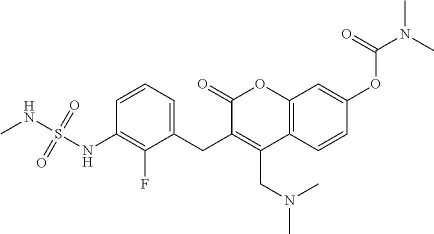 |
| 10 | 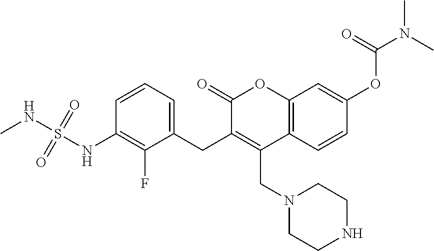 |
| 11 | 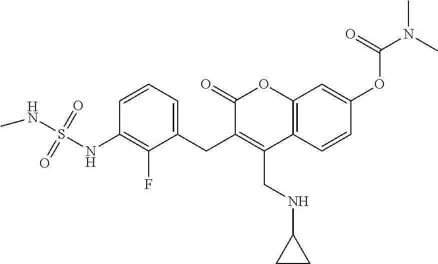 |
| 12 | 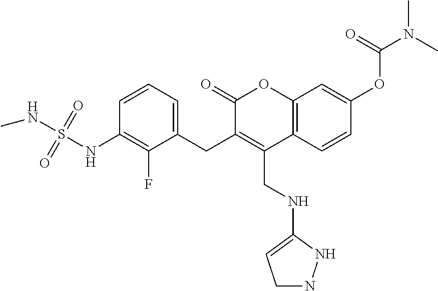 |
| 13 | 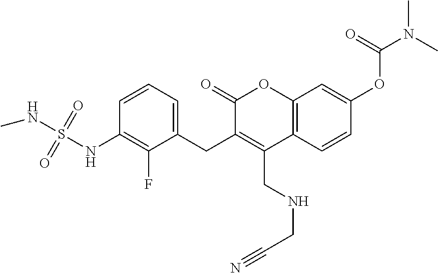 |
| 14 | 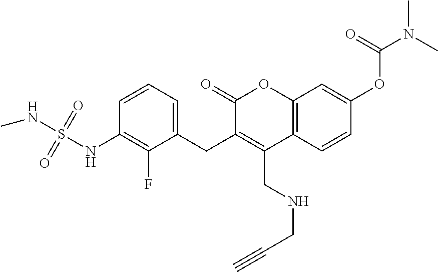 |
| 15 | 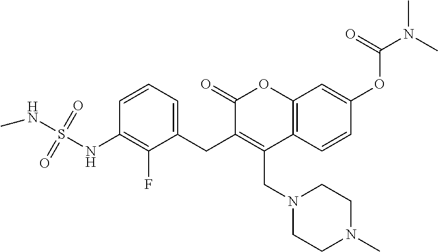 |
| 16 | 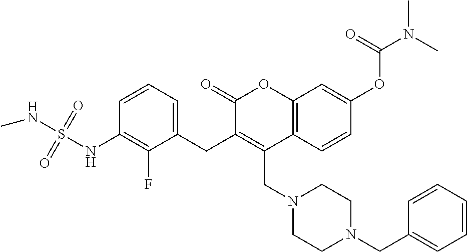 [SMILES: CNS(=O)(=O)Nc1cccc(Cc2c(CN3CCN(Cc4ccccc4)CC3)c3ccc(OC(=O)N(C)C)cc3oc2=O)c1F] |
| 17 | 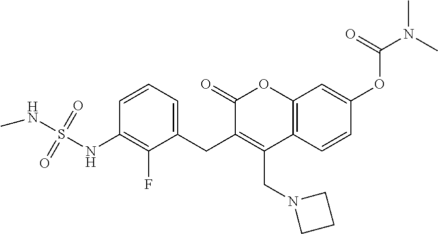 |
| 18 | 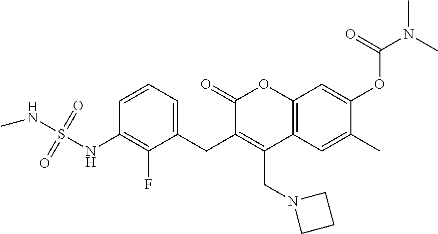 |
| 19 | 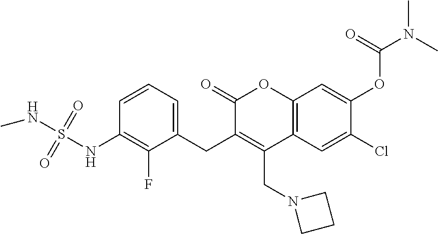 |
| 20 | 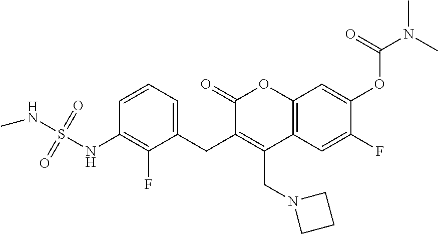 |
| 21 | 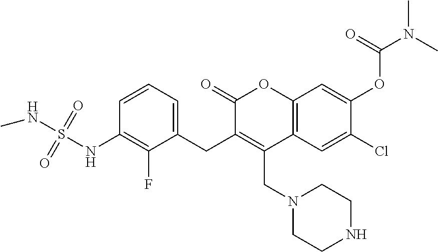 |
| 22 | 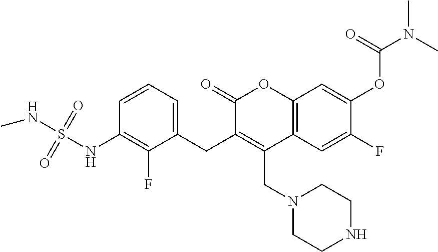 |
| 23 | 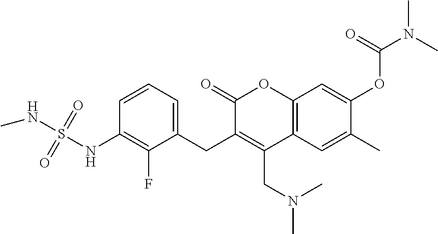 |
| 24 | 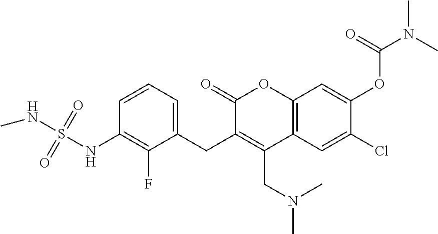 |
| 25 | 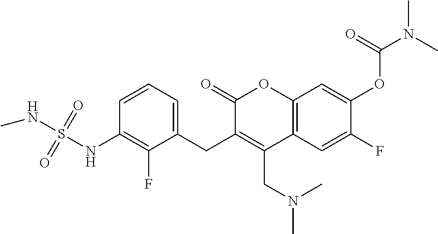 |
| 26 | 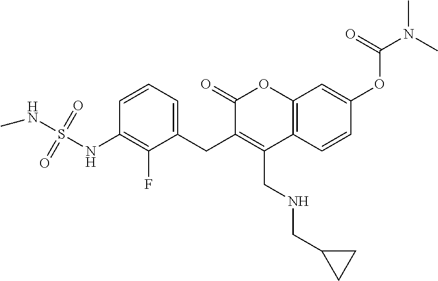 |
| 27 | 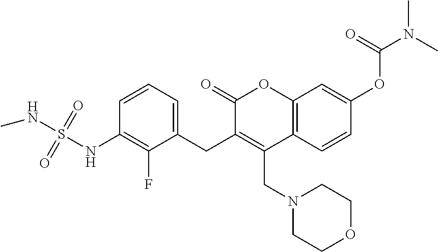 |
| 28 | 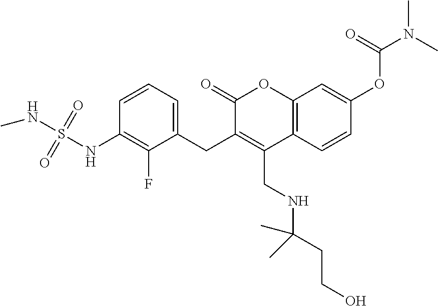 |
| 29 | 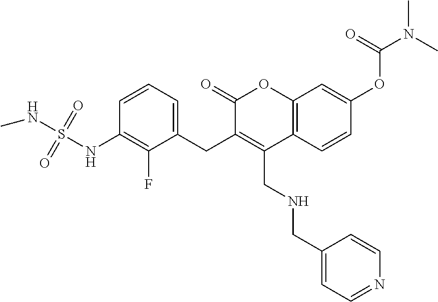 |
| 30 | 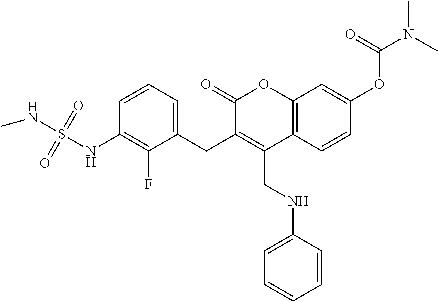 |
| 31 | 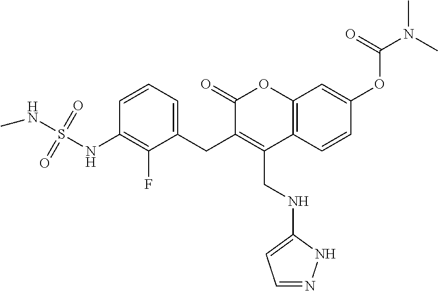 |
| 32 | 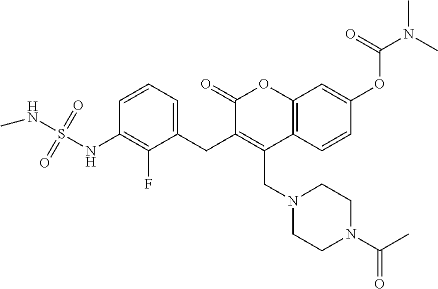 |
| 33 | 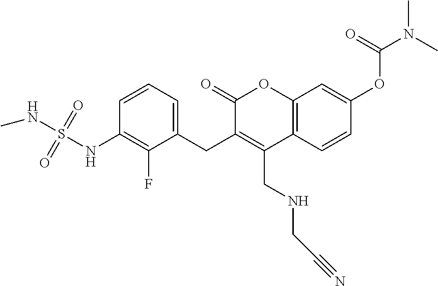 |
| 34 | 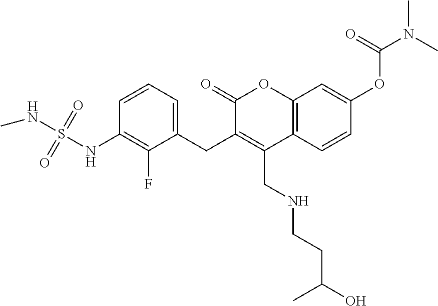 |
| 35 | 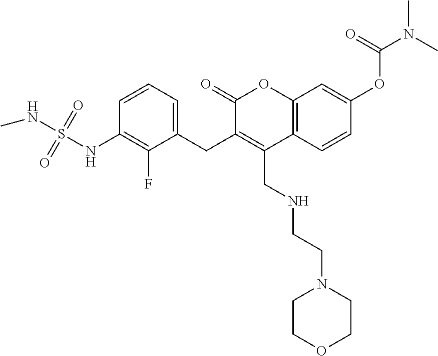 |
| 36 | 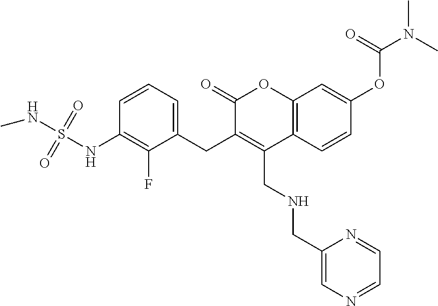 |
| 37 | 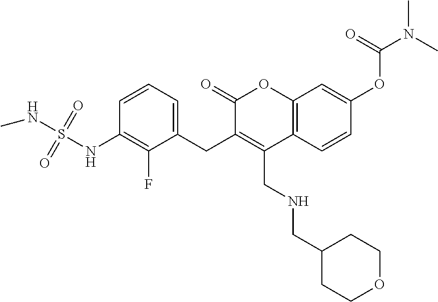 |
| 38 | 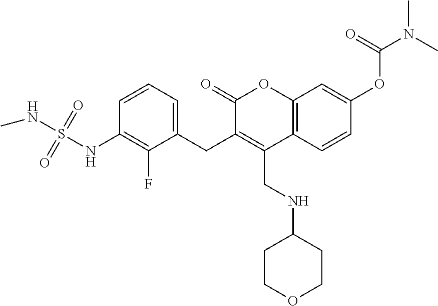 |
| 39 | 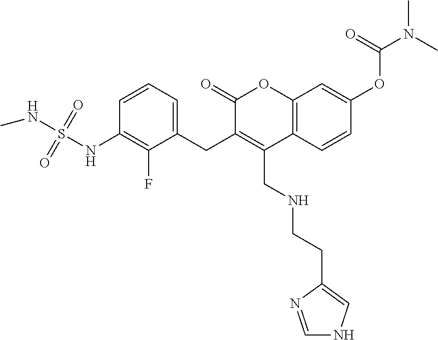 |
| 40 | 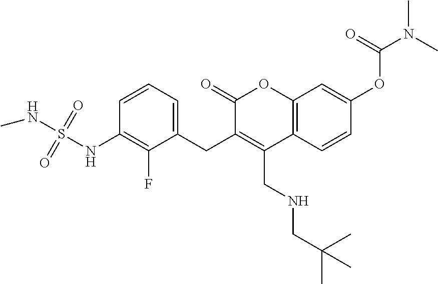 |
| 41 | 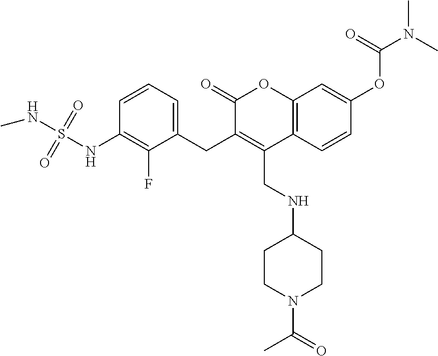 |
| 42 | 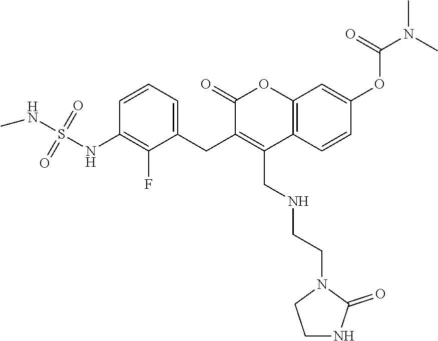 |
| 43 | 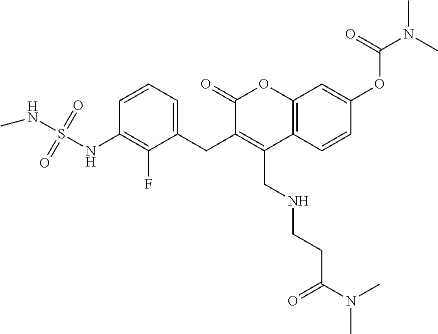 |
| 44 | 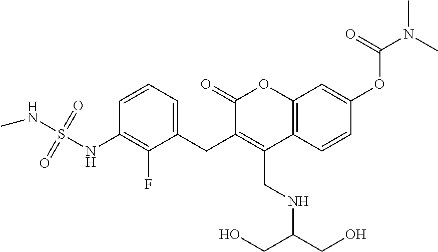 |
| 45 | 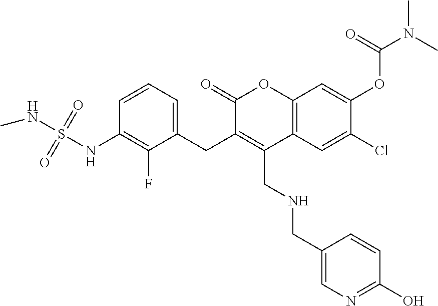 |
| 46 | 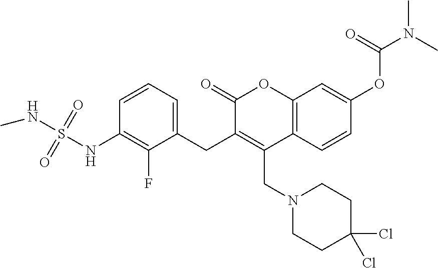 |
| 47 | 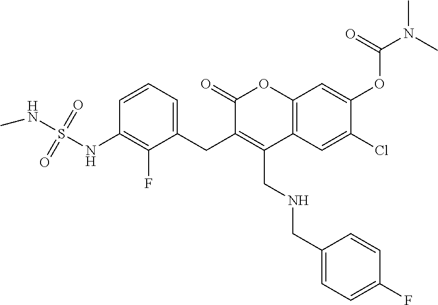 |
| 48 | 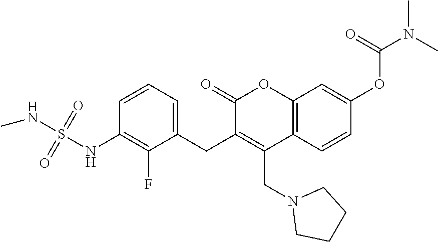 |
| 49 | 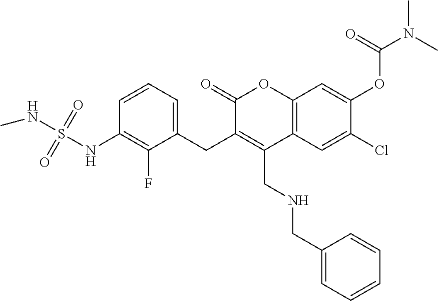 |
| 50 | 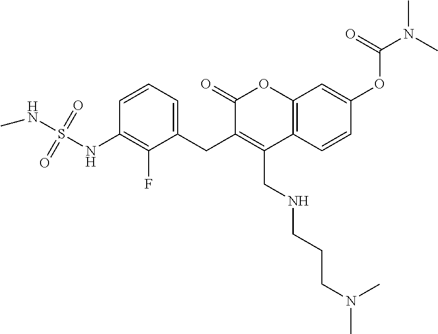 |
| 51 | 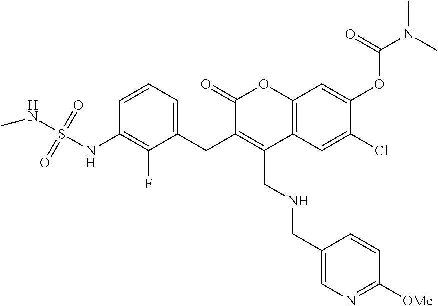 |
| 52 | 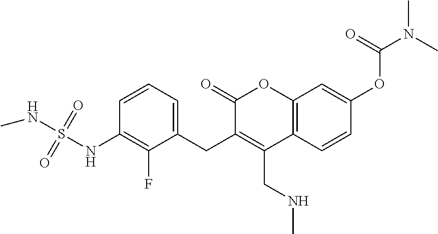 |
| 53 | 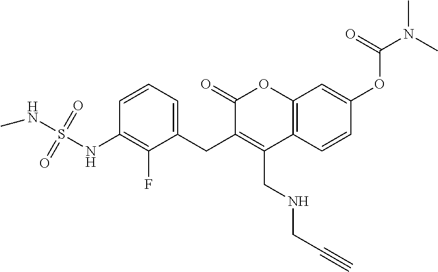 |
| 54 | 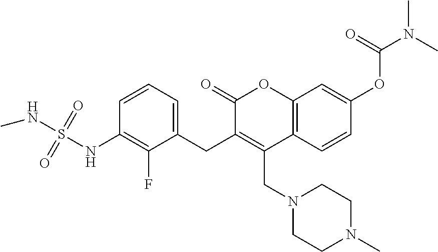 |
| 55 | 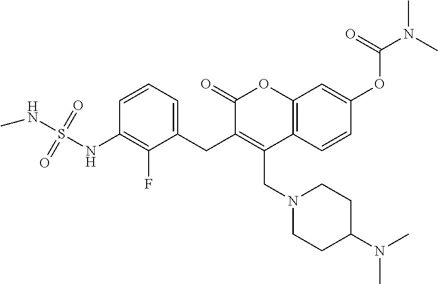 |
| 56 | 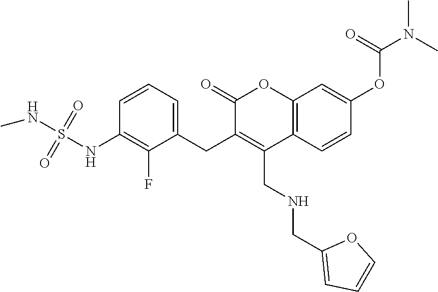 |
| 57 | 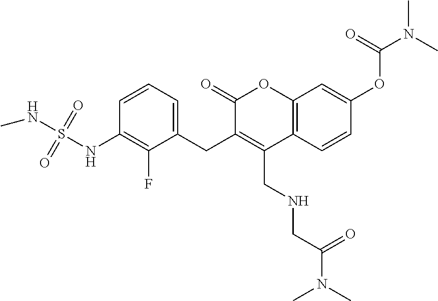 |
| 58 | 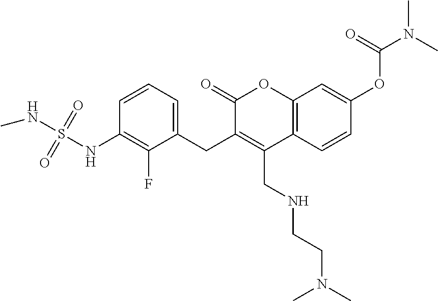 |
| 59 | 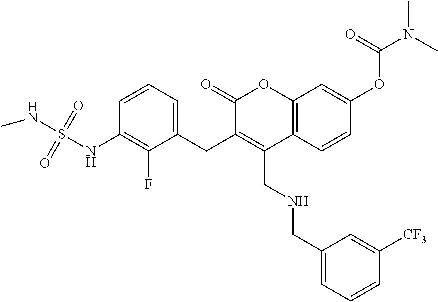 |
| 60 | 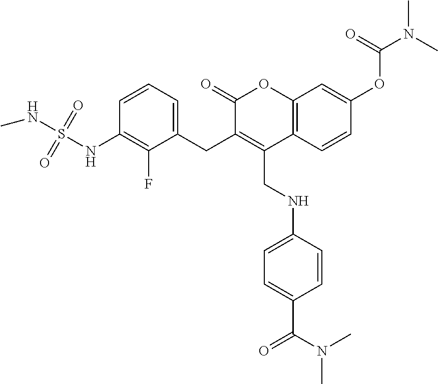 |
| 61 | 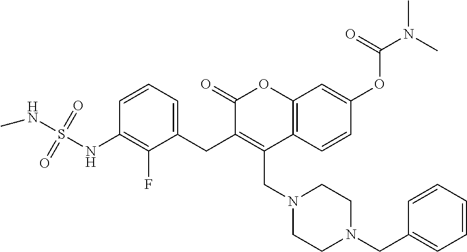 [SMILES: CNS(=O)(=O)Nc1cccc(Cc2c(CN3CCN(Cc4ccccc4)CC3)c3ccc(OC(=O)N(C)C)cc3oc2=O)c1F] |
| 62 | 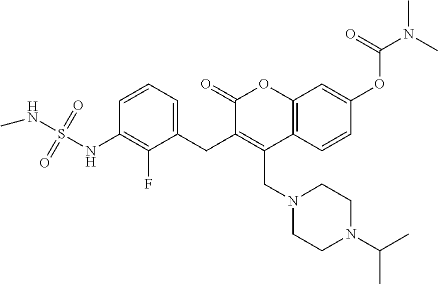 |
| 63 | 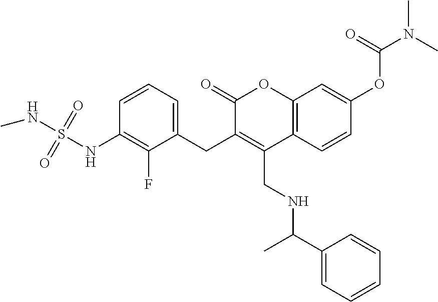 |
| 64 | 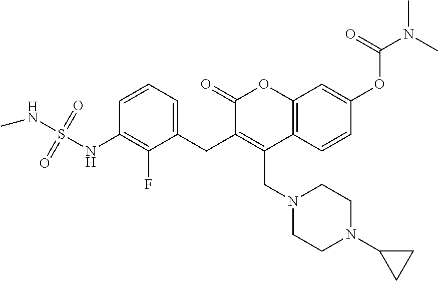 |
| 65 | 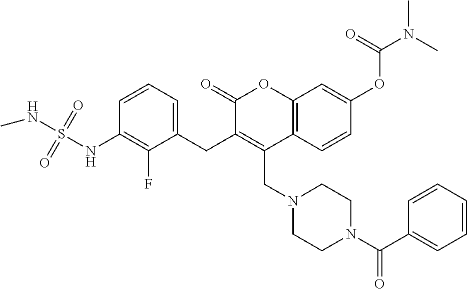 [SMILES: CNS(=O)(=O)Nc1cccc(Cc2c(CN3CCN(C(=O)c4ccccc4)CC3)c3ccc(OC(=O)N(C)C)cc3oc2=O)c1F] |
| 66 | 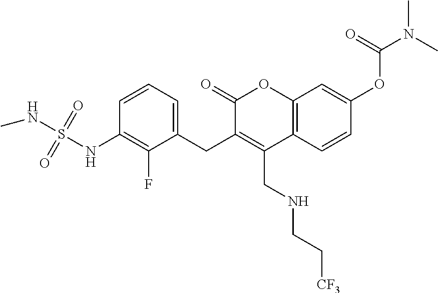 |
| 67 | 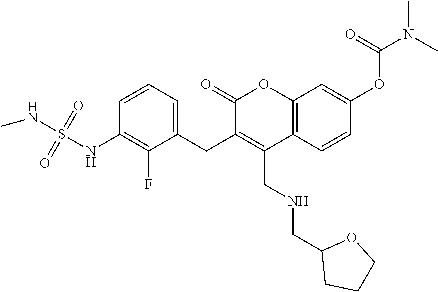 |
| 68 | 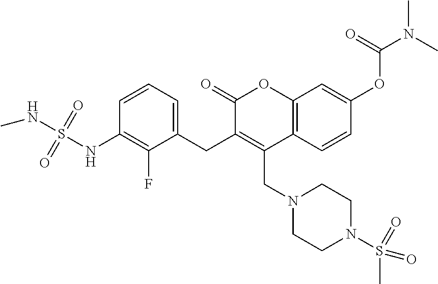 |
| 69 | 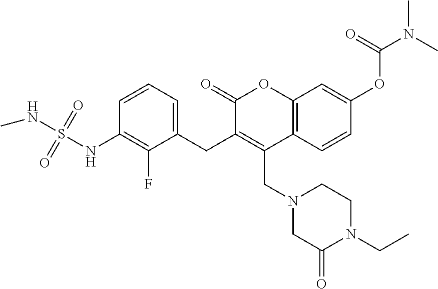 |
| 70 | 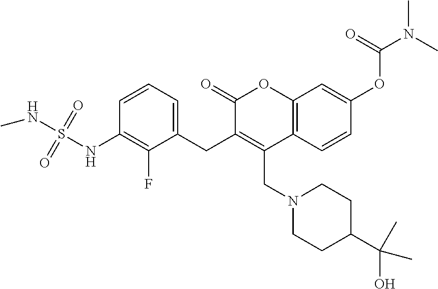 |
| 71 | 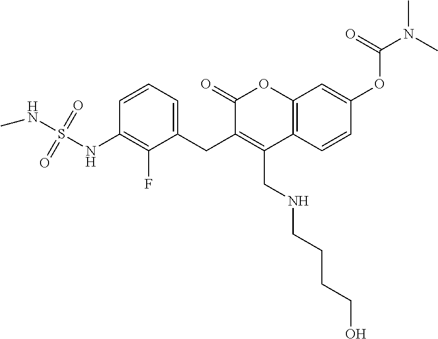 |
| 72 | 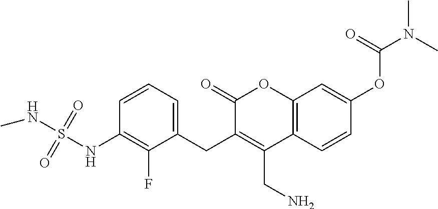 |
| 73 | 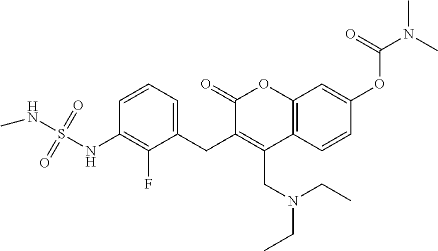 |
| 74 | 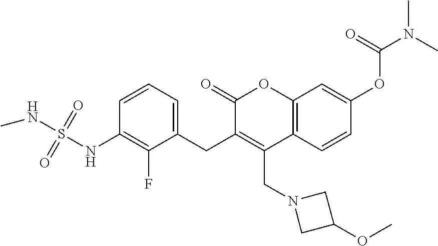 |
| 75 | 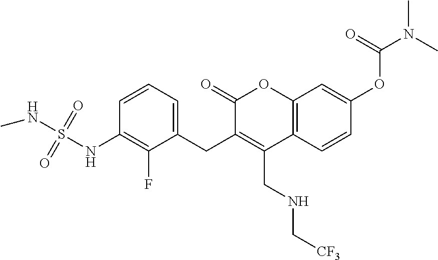 |
| 76 | 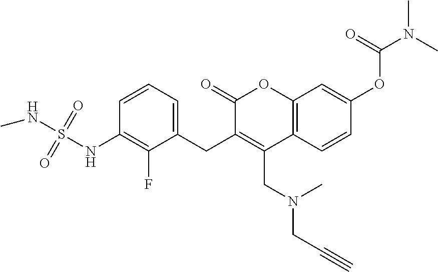 |
| 77 | 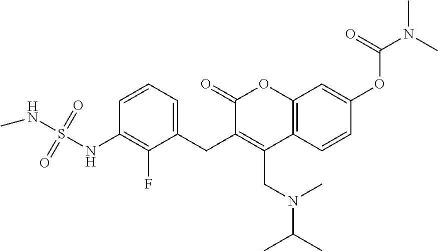 |
| 78 | 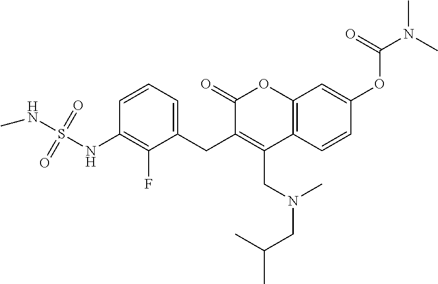 |
| 79 | 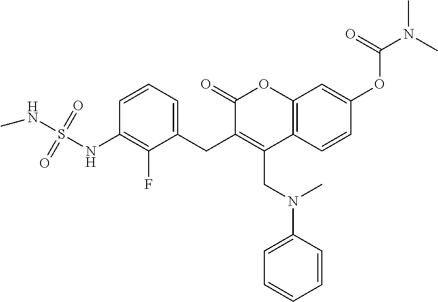 |
| 80 | 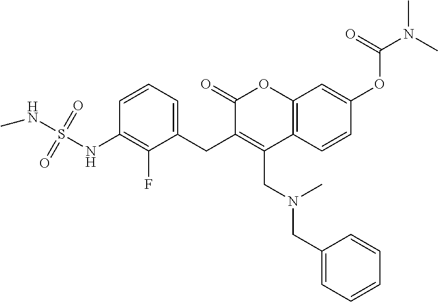 |
| 81 | 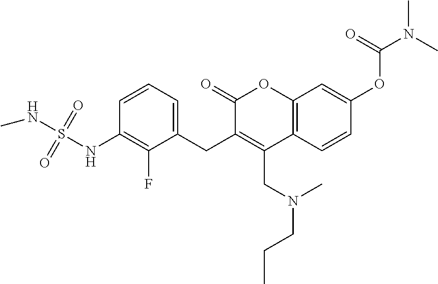 |
| 82 | 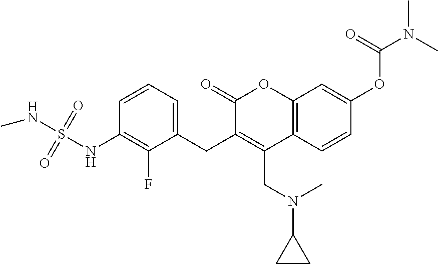 |
| 83 | 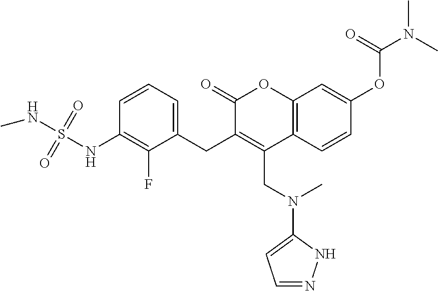 |
| 84 | 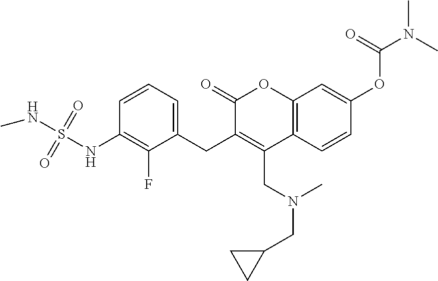 |
| 85 | 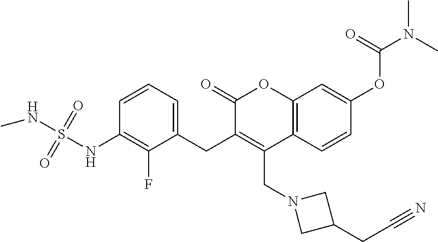 |
| 86 | 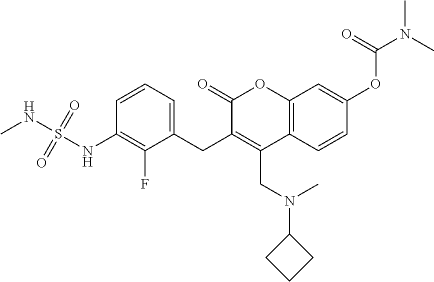 |
| 87 | 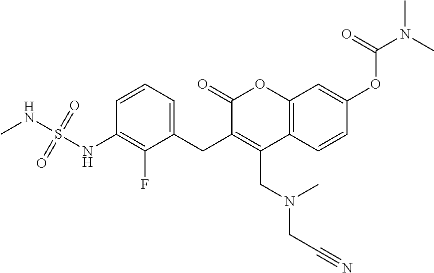 |
| 88 | 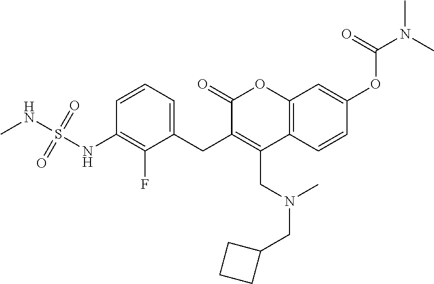 |
| 89 | 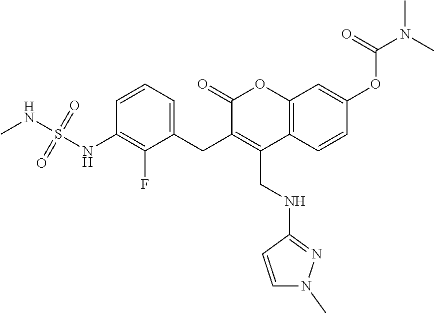 |
| 90 | 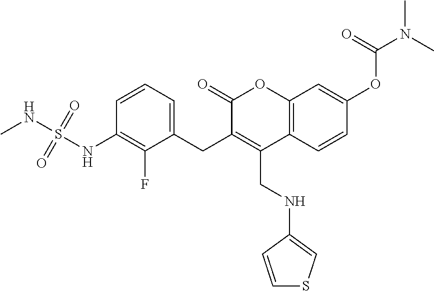 |
| 91 | 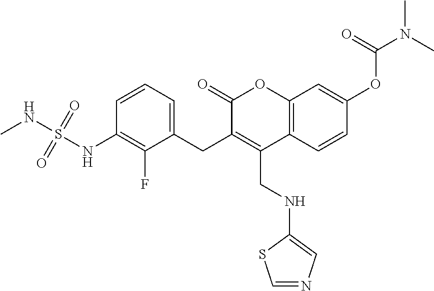 |
| 92 | 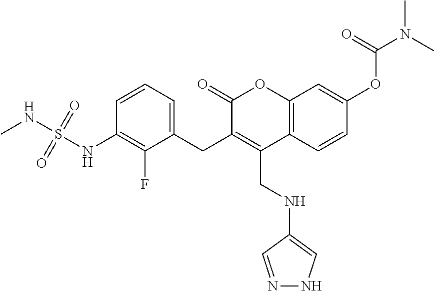 |
| 93 | 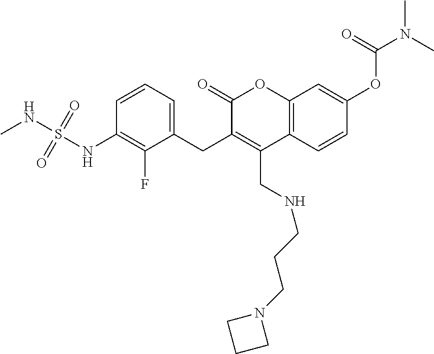 |
| 94 | 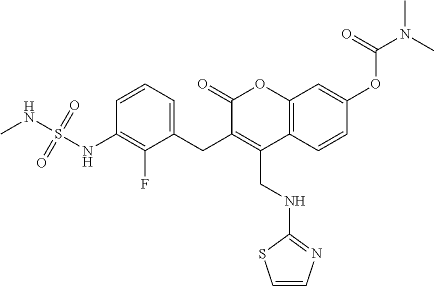 |
| 95 | 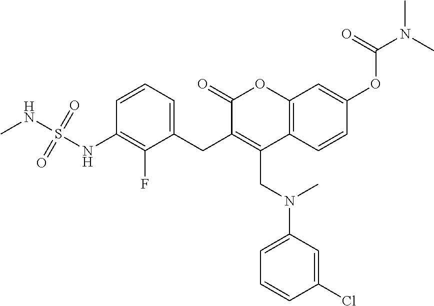 |
| 96 | 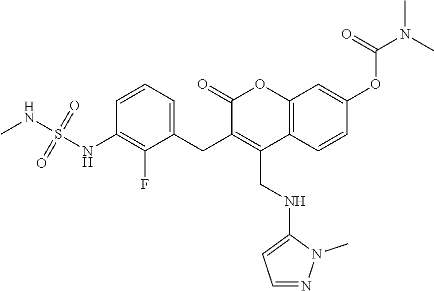 |
| 97 | 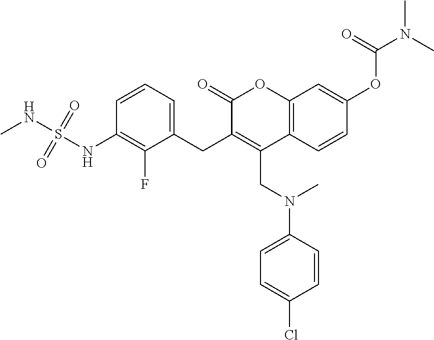 |
| 98 | 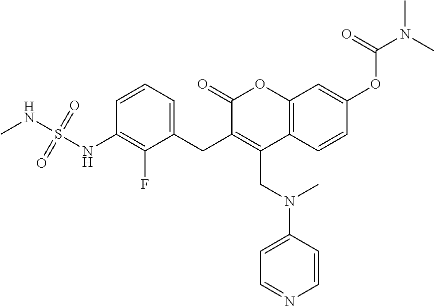 |
| 99 | 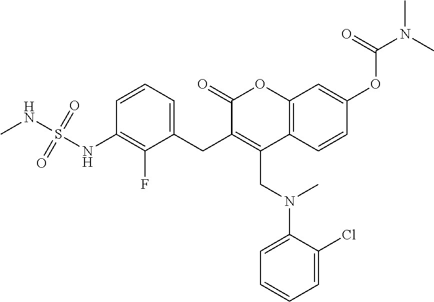 |
| 100 | 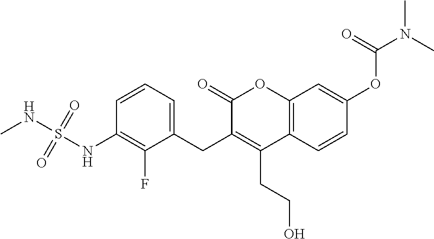 |
| 101 | 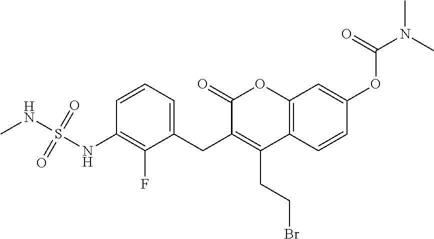 |
| 102 | 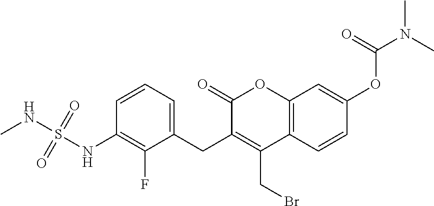 |
| 103 | 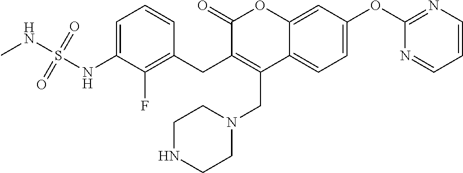 [SMILES: CNS(=O)(=O)Nc1cccc(Cc2c(CN3CCNCC3)c3ccc(Oc4ncccn4)cc3oc2=O)c1F] |
| 104 | 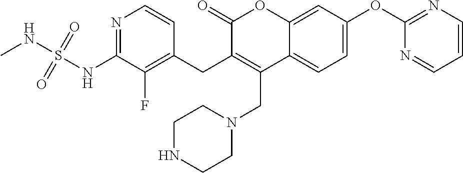 [SMILES: CNS(=O)(=O)Nc1nccc(Cc2c(CN3CCNCC3)c3ccc(Oc4ncccn4)cc3oc2=O)c1F] |
| 105 | 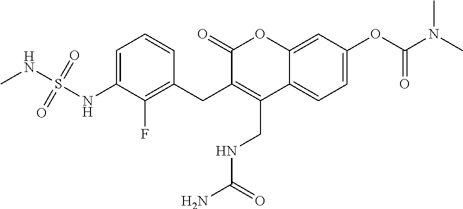 |
| 106 | 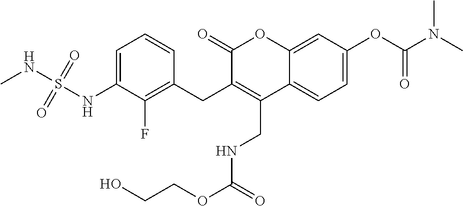 |
| 107 | 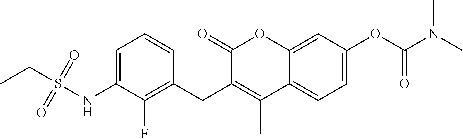 [SMILES: CCS(=O)(=O)Nc1cccc(Cc2c(C)c3ccc(OC(=O)N(C)C)cc3oc2=O)c1F] |
| 108 | 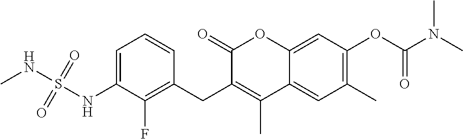 |
| 109 | 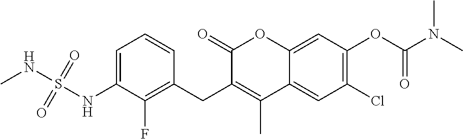 |
| 110 | 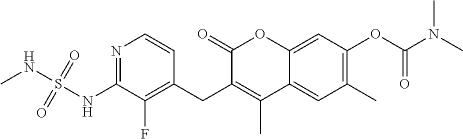 |
| 111 | 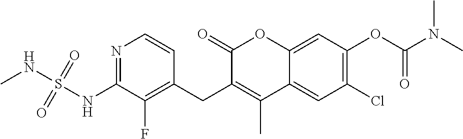 |
| 112 | 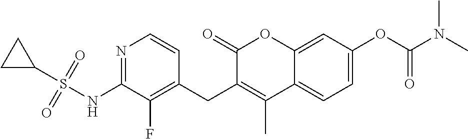 [SMILES: Cc1c(Cc2ccnc(NS(=O)(=O)C3CC3)c2F)c(=O)oc2cc(OC(=O)N(C)C)ccc12] |
| 113 | 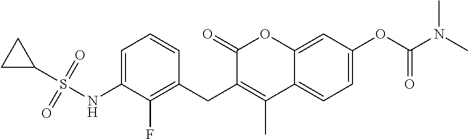 [SMILES: Cc1c(Cc2cccc(NS(=O)(=O)C3CC3)c2F)c(=O)oc2cc(OC(=O)N(C)C)ccc12] |
| 114 | 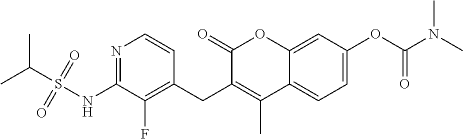 [SMILES: Cc1c(Cc2ccnc(NS(=O)(=O)C(C)C)c2F)c(=O)oc2cc(OC(=O)N(C)C)ccc12] |
| 115 | 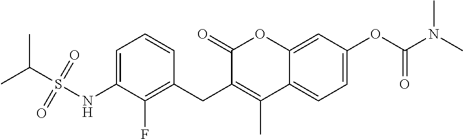 [SMILES: Cc1c(Cc2cccc(NS(=O)(=O)C(C)C)c2F)c(=O)oc2cc(OC(=O)N(C)C)ccc12] |
| 116 | 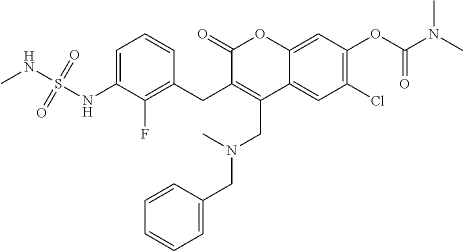 |
| 117 | 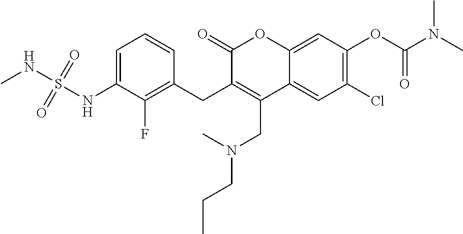 |
| 118 | 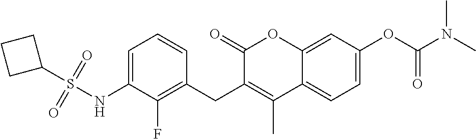 [SMILES: Cc1c(Cc2cccc(NS(=O)(=O)C3CCC3)c2F)c(=O)oc2cc(OC(=O)N(C)C)ccc12] |
| 119 | 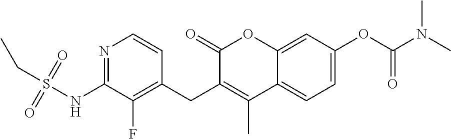 [SMILES: CCS(=O)(=O)Nc1nccc(Cc2c(C)c3ccc(OC(=O)N(C)C)cc3oc2=O)c1F] |
| 120 | 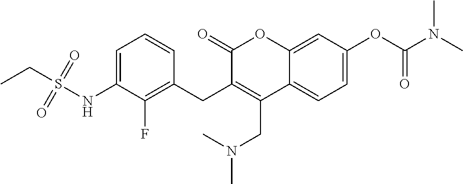 [SMILES: CCS(=O)(=O)Nc1cccc(Cc2c(CN(C)C)c3ccc(OC(=O)N(C)C)cc3oc2=O)c1F] |
| 121 | 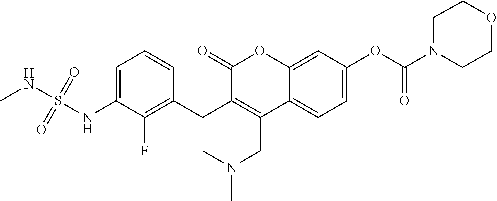 [SMILES: CNS(=O)(=O)Nc1cccc(Cc2c(CN(C)C)c3ccc(OC(=O)N4CCOCC4)cc3oc2=O)c1F] |
| 122 | 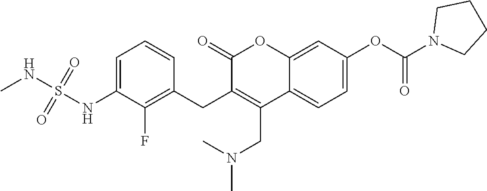 [SMILES: CNS(=O)(=O)Nc1cccc(Cc2c(CN(C)C)c3ccc(OC(=O)N4CCCC4)cc3oc2=O)c1F] |
| 123 | 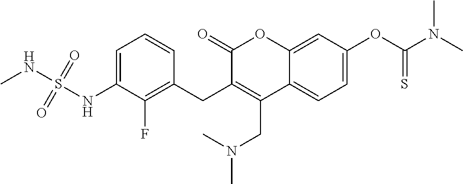 |
| 124 | 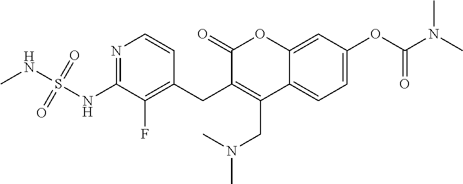 |
| 125 | 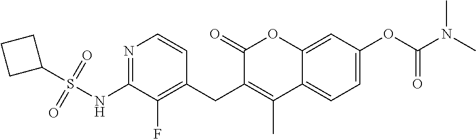 [SMILES: Cc1c(Cc2ccnc(NS(=O)(=O)C3CCC3)c2F)c(=O)oc2cc(OC(=O)N(C)C)ccc12] |
| 126 | 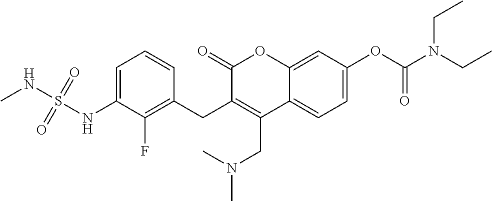 [SMILES: CCN(CC)C(=O)Oc1ccc2c(CN(C)C)c(Cc3cccc(NS(=O)(=O)NC)c3F)c(=O)oc2c1] |
| 127 | 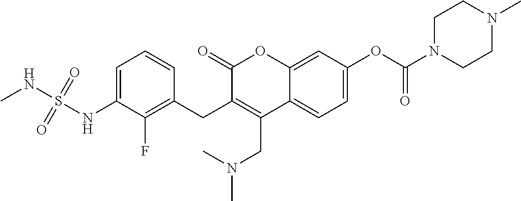 |
| 128 | 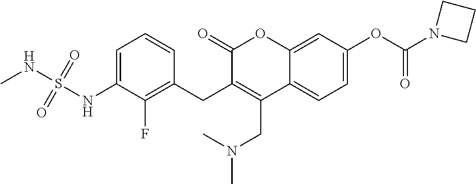 [SMILES: CNS(=O)(=O)Nc1cccc(Cc2c(CN(C)C)c3ccc(OC(=O)N4CCC4)cc3oc2=O)c1F] |
| 129 | 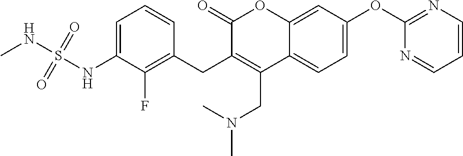 [SMILES: CNS(=O)(=O)Nc1cccc(Cc2c(CN(C)C)c3ccc(Oc4ncccn4)cc3oc2=O)c1F] |
| 130 | 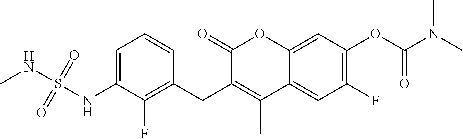 |
| 131 | 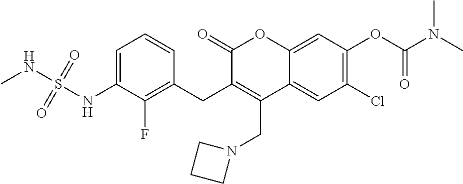 |
| 132 | 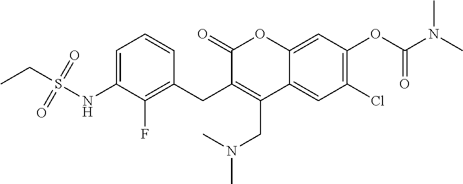 [SMILES: CCS(=O)(=O)Nc1cccc(Cc2c(CN(C)C)c3cc(Cl)c(OC(=O)N(C)C)cc3oc2=O)c1F] |
| 133 | 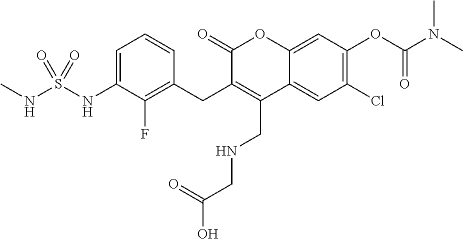 |
| 134 | 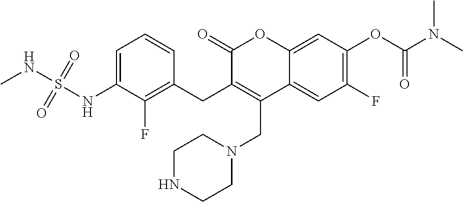 |
| 135 | 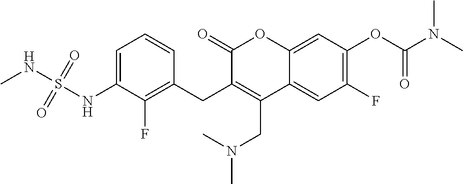 |
| 136 | 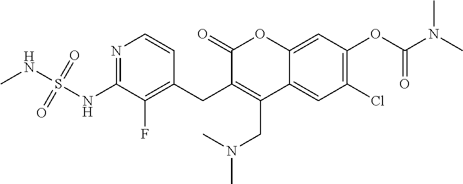 |
| 137 | 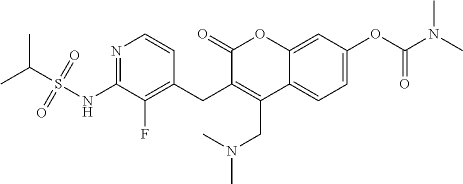 [SMILES: CC(C)S(=O)(=O)Nc1nccc(Cc2c(CN(C)C)c3ccc(OC(=O)N(C)C)cc3oc2=O)c1F] |
| 138 | 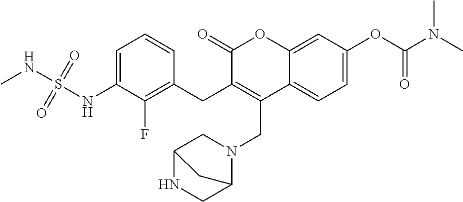 |
| 139 | 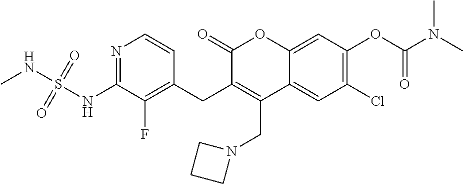 |
| 140 | 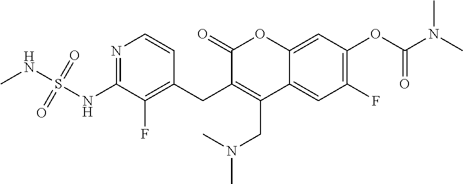 |
| 141 | 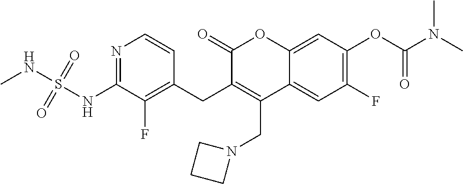 |
| 142 | 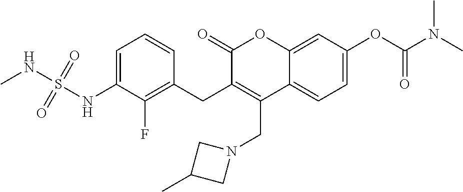 |
| 143 | 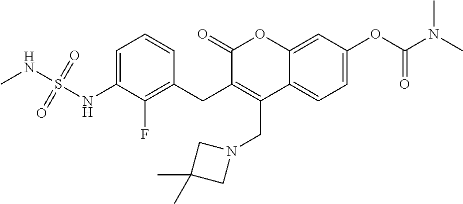 |
| 144 | 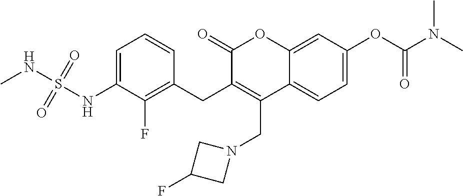 |
| 145 | 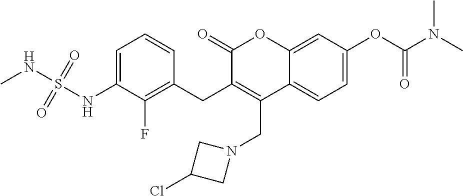 |
| 146 | 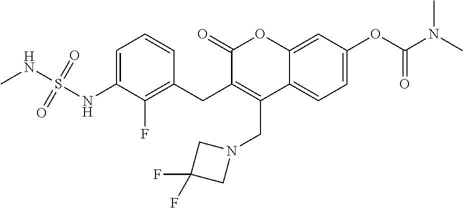 |
| 147 | 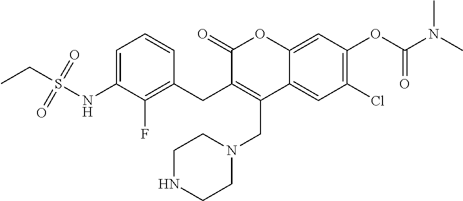 [SMILES: CCS(=O)(=O)Nc1cccc(Cc2c(CN3CCNCC3)c3cc(Cl)c(OC(=O)N(C)C)cc3oc2=O)c1F] |
| 148 | 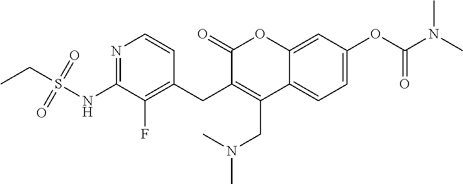 [SMILES: CCS(=O)(=O)Nc1nccc(Cc2c(CN(C)C)c3ccc(OC(=O)N(C)C)cc3oc2=O)c1F] |
| 149 | 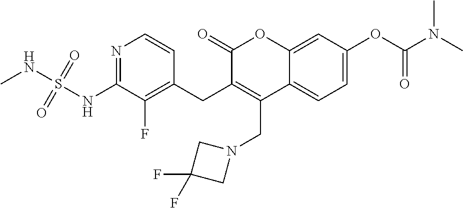 |
| 150 | 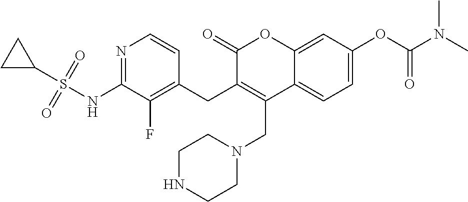 [SMILES: CN(C)C(=O)Oc1ccc2c(CN3CCNCC3)c(Cc3ccnc(NS(=O)(=O)C4CC4)c3F)c(=O)oc2c1] |
| 151 | 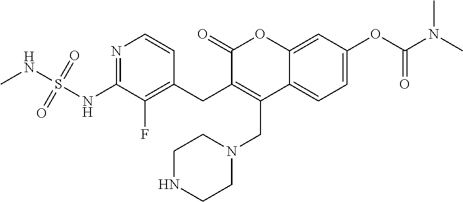 |
| 152 | 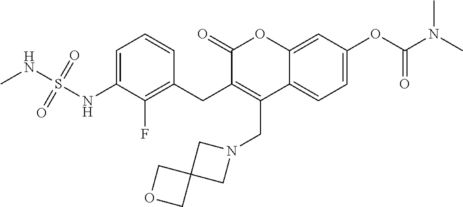 |
| 153 | 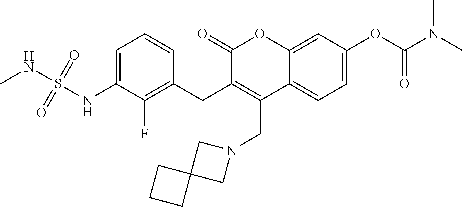 |
| 154 | 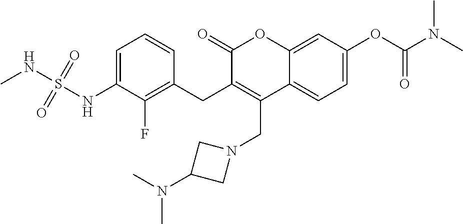 |
| 155 | 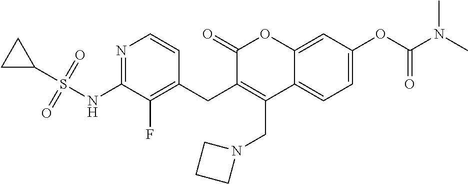 [SMILES: CN(C)C(=O)Oc1ccc2c(CN3CCC3)c(Cc3ccnc(NS(=O)(=O)C4CC4)c3F)c(=O)oc2c1] |
| 156 | 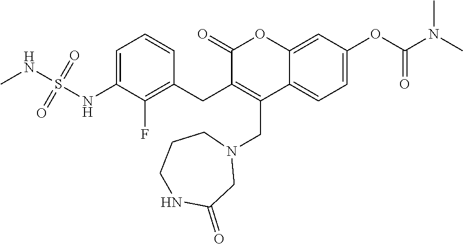 |
| 157 | 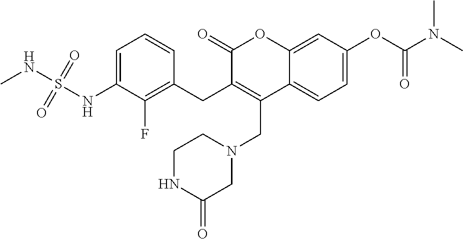 |
| 158 | 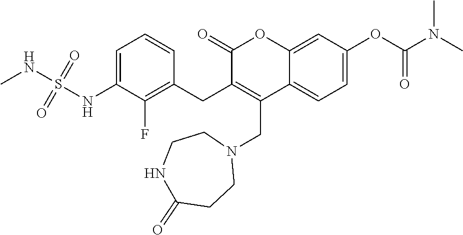 |
| 159 | 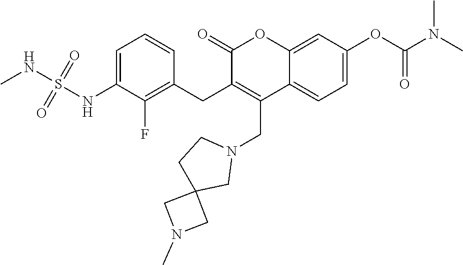 |
| 160 | 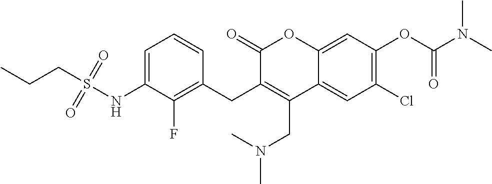 [SMILES: CCCS(=O)(=O)Nc1cccc(Cc2c(CN(C)C)c3cc(Cl)c(OC(=O)N(C)C)cc3oc2=O)c1F] |
| 161 | 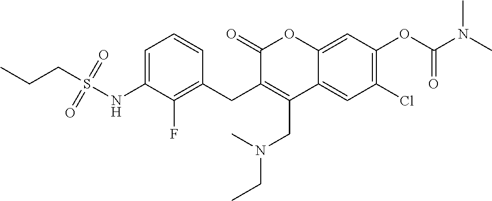 [SMILES: CCCS(=O)(=O)Nc1cccc(Cc2c(CN(C)CC)c3cc(Cl)c(OC(=O)N(C)C)cc3oc2=O)c1F] |
| 162 | 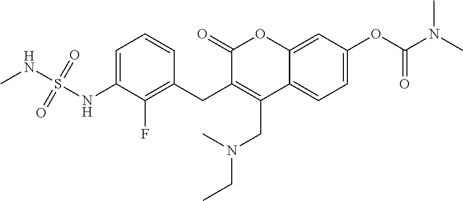 |
| 163 | 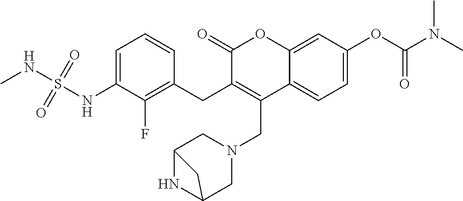 |
| 164 | 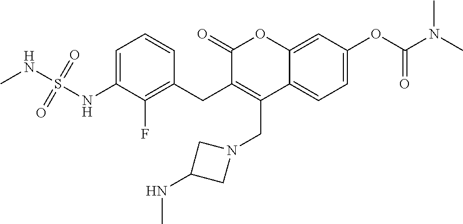 |
| 165 | 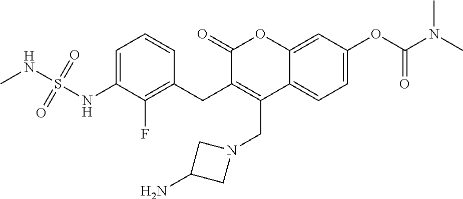 |
| 166 | 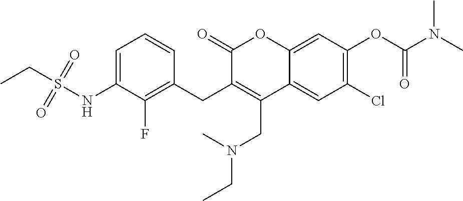 [SMILES: CCN(C)Cc1c(Cc2cccc(NS(=O)(=O)CC)c2F)c(=O)oc2cc(OC(=O)N(C)C)c(Cl)cc12] |
| 167 | 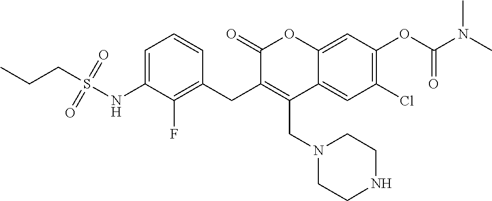 [SMILES: CCCS(=O)(=O)Nc1cccc(Cc2c(CN3CCNCC3)c3cc(Cl)c(OC(=O)N(C)C)cc3oc2=O)c1F] |
| 168 | 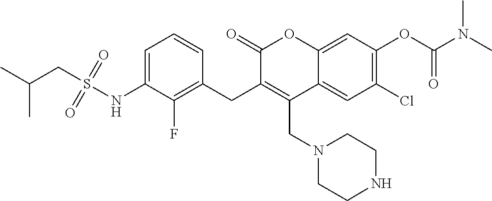 [SMILES: CC(C)CS(=O)(=O)Nc1cccc(Cc2c(CN3CCNCC3)c3cc(Cl)c(OC(=O)N(C)C)cc3oc2=O)c1F] |
| 169 | 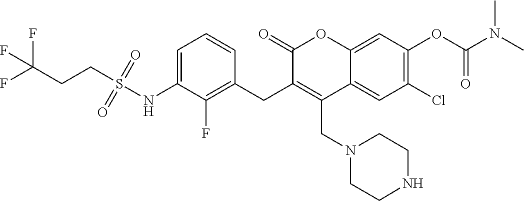 [SMILES: CN(C)C(=O)Oc1cc2oc(=O)c(Cc3cccc(NS(=O)(=O)CCC(F)(F)F)c3F)c(CN3CCNCC3)c2cc1Cl] |
| 170 | 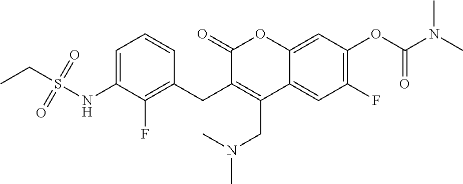 [SMILES: CCS(=O)(=O)Nc1cccc(Cc2c(CN(C)C)c3cc(F)c(OC(=O)N(C)C)cc3oc2=O)c1F] |
| 171 | 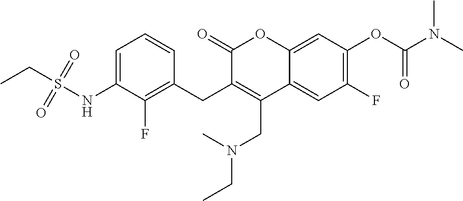 [SMILES: CCN(C)Cc1c(Cc2cccc(NS(=O)(=O)CC)c2F)c(=O)oc2cc(OC(=O)N(C)C)c(F)cc12] |
| 172 | 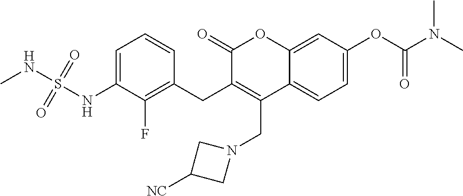 |
| 173 | 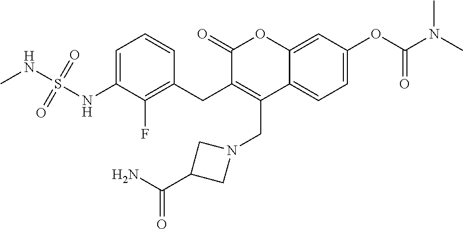 |
| 174 | 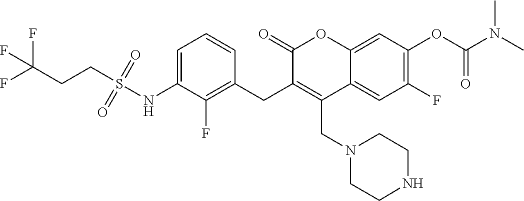 [SMILES: CN(C)C(=O)Oc1cc2oc(=O)c(Cc3cccc(NS(=O)(=O)CCC(F)(F)F)c3F)c(CN3CCNCC3)c2cc1F] |
| 175 | 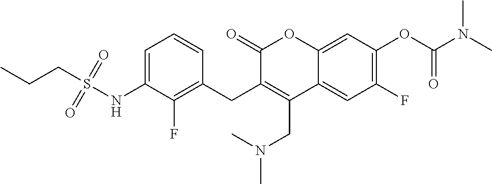 [SMILES: CCCS(=O)(=O)Nc1cccc(Cc2c(CN(C)C)c3cc(F)c(OC(=O)N(C)C)cc3oc2=O)c1F] |
| 176 | 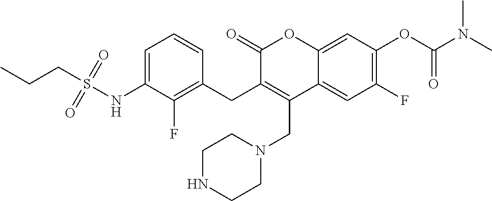 [SMILES: CCCS(=O)(=O)Nc1cccc(Cc2c(CN3CCNCC3)c3cc(F)c(OC(=O)N(C)C)cc3oc2=O)c1F] |
| 177 | 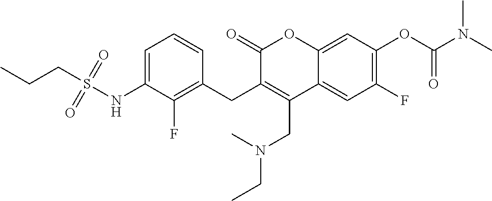 [SMILES: CCCS(=O)(=O)Nc1cccc(Cc2c(CN(C)CC)c3cc(F)c(OC(=O)N(C)C)cc3oc2=O)c1F] |
| 178 | 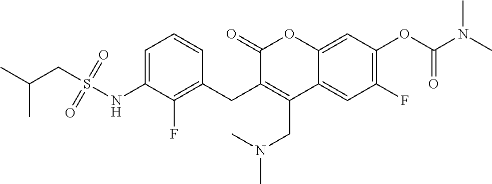 [SMILES: CC(C)CS(=O)(=O)Nc1cccc(Cc2c(CN(C)C)c3cc(F)c(OC(=O)N(C)C)cc3oc2=O)c1F] |
| 179 | 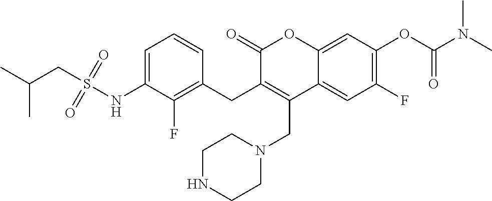 [SMILES: CC(C)CS(=O)(=O)Nc1cccc(Cc2c(CN3CCNCC3)c3cc(F)c(OC(=O)N(C)C)cc3oc2=O)c1F] |
| 180 | 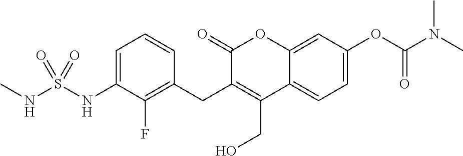 |
| 181 | 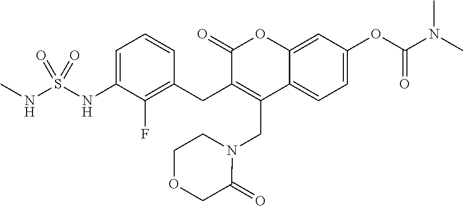 |
| 182 | 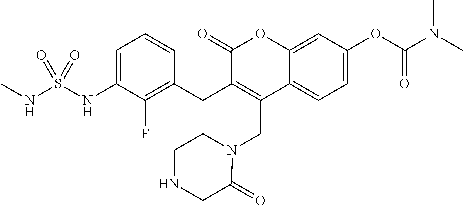 |
| 183 | 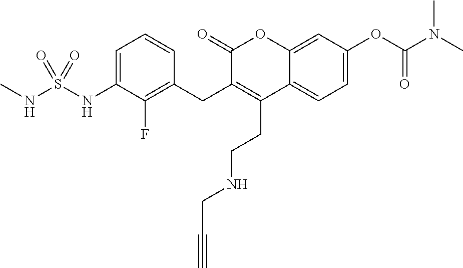 |
| 184 | 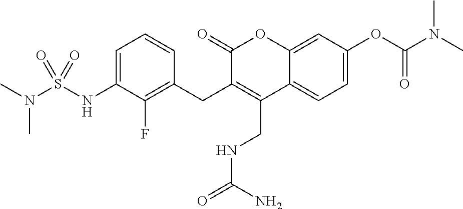 |
| 185 | 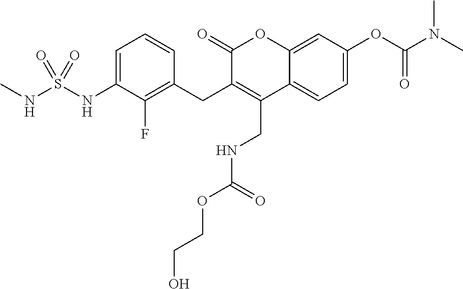 |
| 186 | 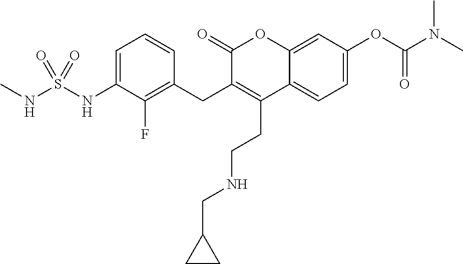 |
| 187 | 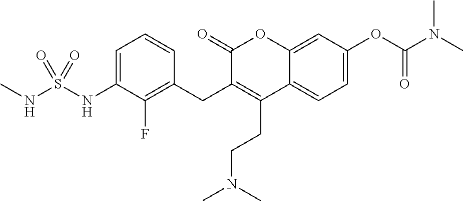 |
| 188 | 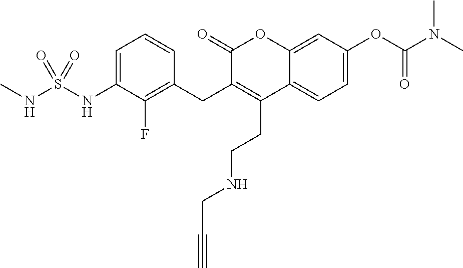 |
| 189 | 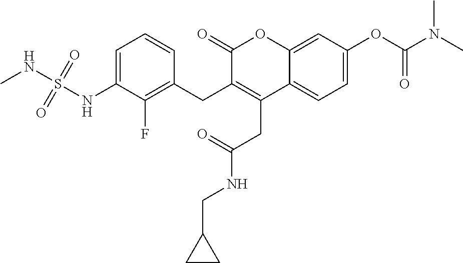 |
| 190 | 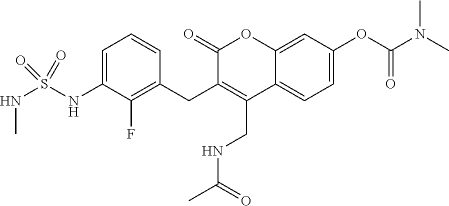 [SMILES: CNS(=O)(=O)Nc1cccc(Cc2c(CNC(C)=O)c3ccc(OC(=O)N(C)C)cc3oc2=O)c1F] |
| 191 | 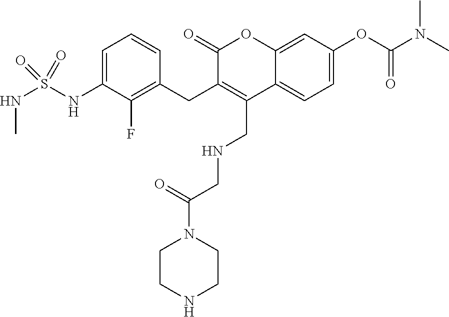 [SMILES: CNS(=O)(=O)Nc1cccc(Cc2c(CNCC(=O)N3CCNCC3)c3ccc(OC(=O)N(C)C)cc3oc2=O)c1F] |
| 192 | 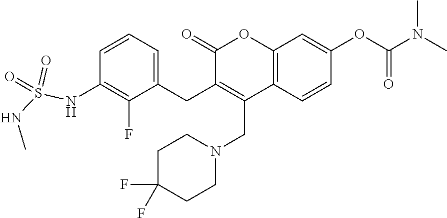 [SMILES: CNS(=O)(=O)Nc1cccc(Cc2c(CN3CCC(F)(F)CC3)c3ccc(OC(=O)N(C)C)cc3oc2=O)c1F] |
| 193 | 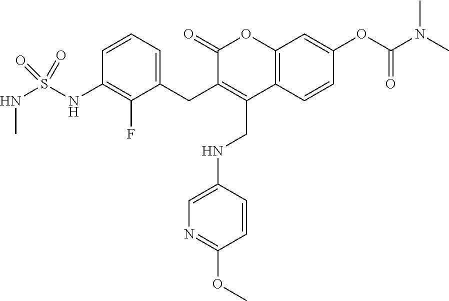 [SMILES: CNS(=O)(=O)Nc1cccc(Cc2c(CNc3ccc(OC)nc3)c3ccc(OC(=O)N(C)C)cc3oc2=O)c1F] |
| 194 | 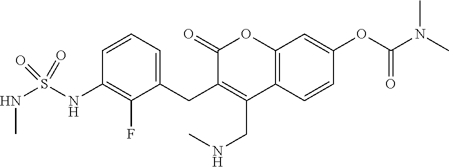 [SMILES: CNCc1c(Cc2cccc(NS(=O)(=O)NC)c2F)c(=O)oc2cc(OC(=O)N(C)C)ccc12] |
| 195 | 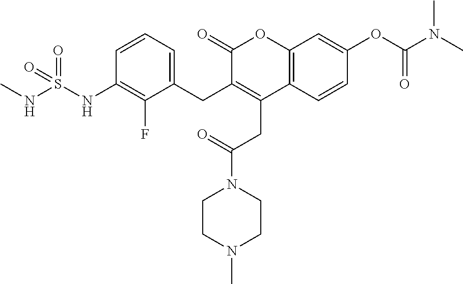 |
| 196 | 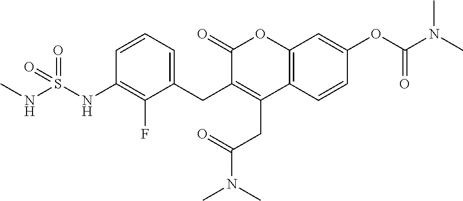 |
| 197 | 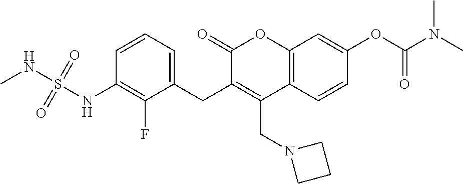 |
| 198 | 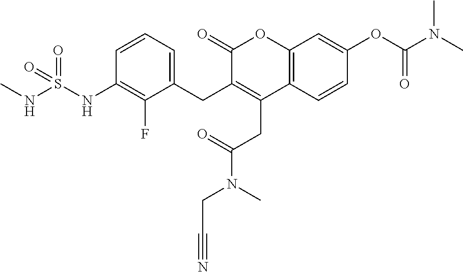 |
| 199 | 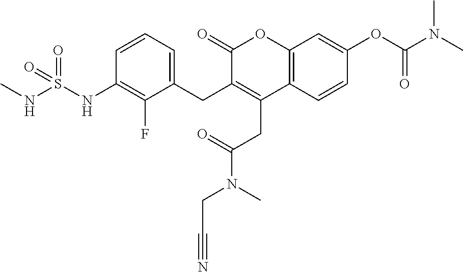 |
| 200 | 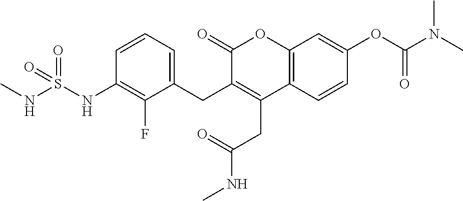 |
| 201 | 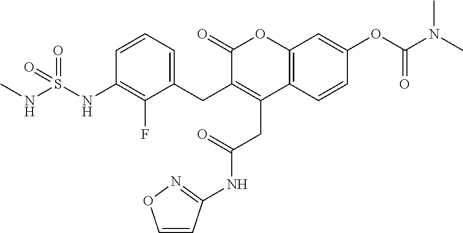 |
| 202 | 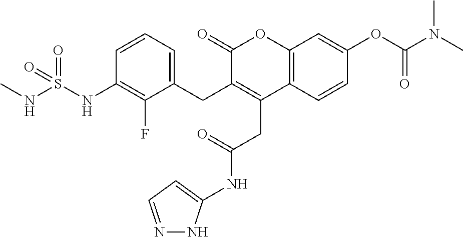 |
| 203 | 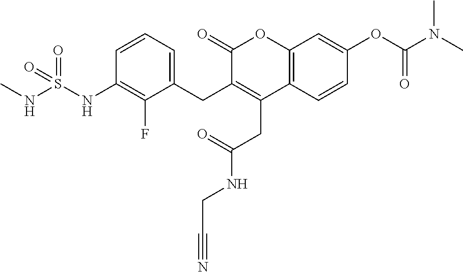 |
| 204 | 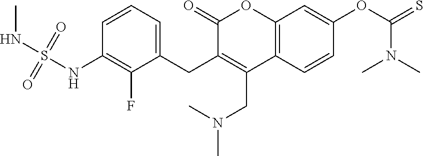 [SMILES: CNS(=O)(=O)Nc1cccc(Cc2c(CN(C)C)c3ccc(OC(=S)N(C)C)cc3oc2=O)c1F] |
| 205 | 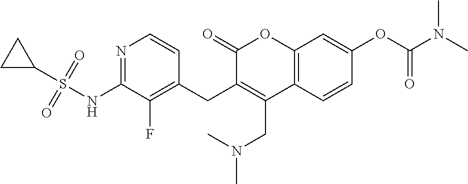 [SMILES: CN(C)Cc1c(Cc2ccnc(NS(=O)(=O)C3CC3)c2F)c(=O)oc2cc(OC(=O)N(C)C)ccc12] |
| 206 | 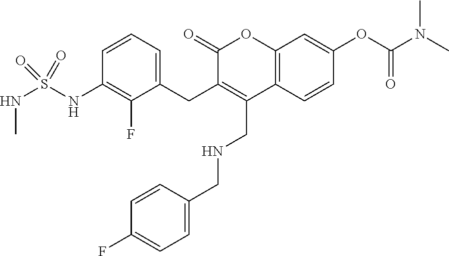 [SMILES: CNS(=O)(=O)Nc1cccc(Cc2c(CNCc3ccc(F)cc3)c3ccc(OC(=O)N(C)C)cc3oc2=O)c1F] |
| 207 | 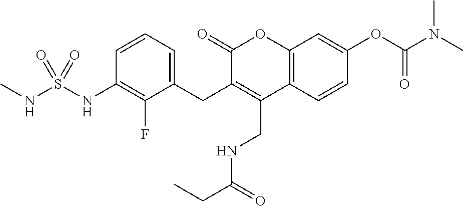 |
| 208 | 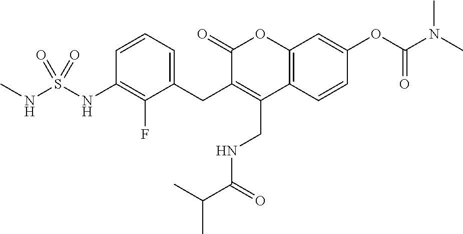 |
| 209 | 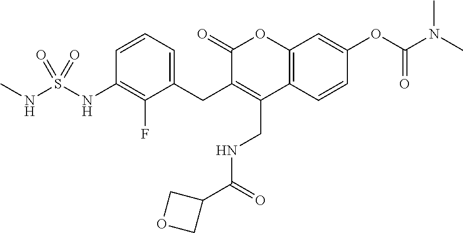 |
| 210 | 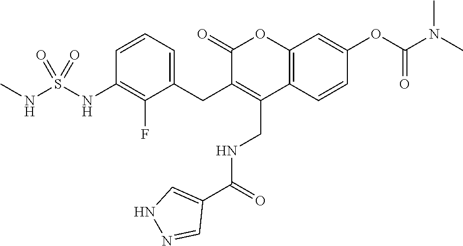 |
| 211 | 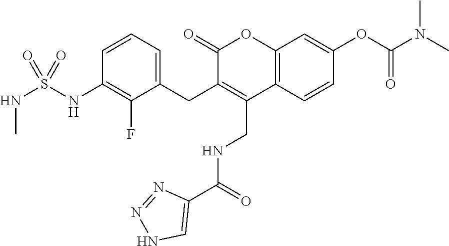 [SMILES: CNS(=O)(=O)Nc1cccc(Cc2c(CNC(=O)c3c[nH]nn3)c3ccc(OC(=O)N(C)C)cc3oc2=O)c1F] |
| 212 | 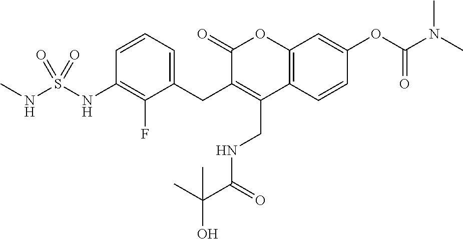 |
| 213 | 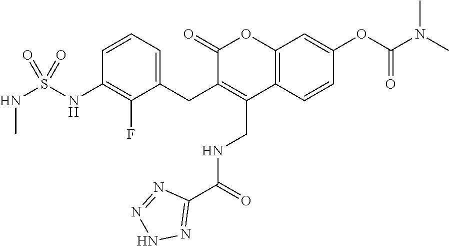 [SMILES: CNS(=O)(=O)Nc1cccc(Cc2c(CNC(=O)c3nn[nH]n3)c3ccc(OC(=O)N(C)C)cc3oc2=O)c1F] |
| 214 | 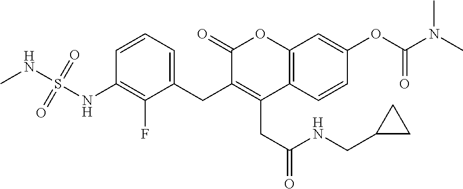 |
| 215 | 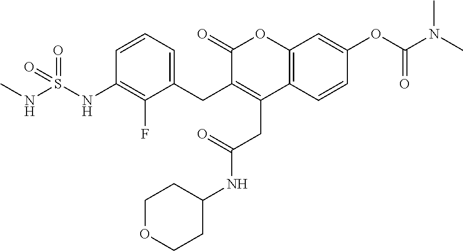 |
| 216 | 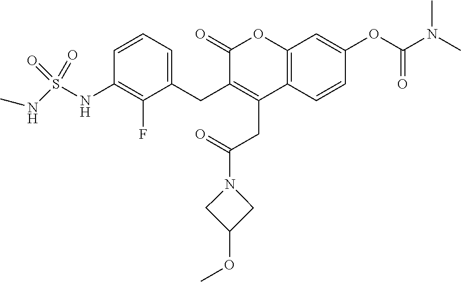 |
| 217 | 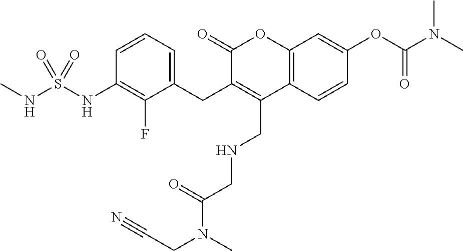 |
| 218 | 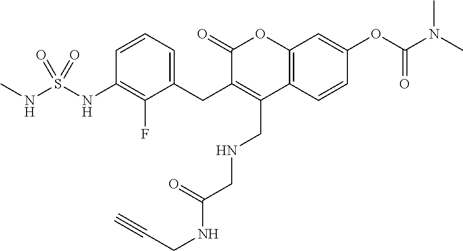 |
| 219 | 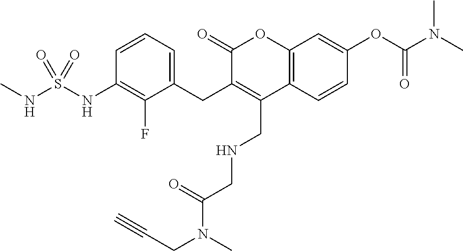 |
| 220 | 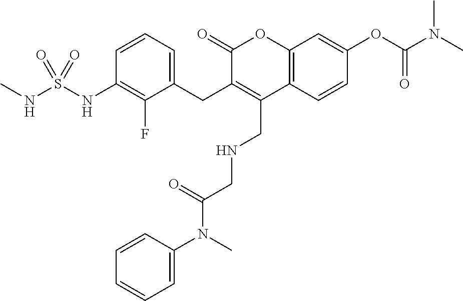 |
| 221 | 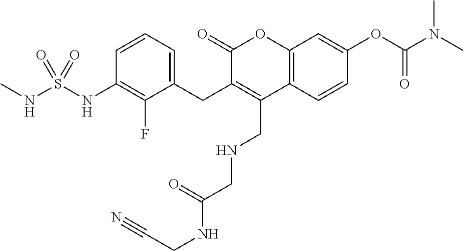 |
| 222 | 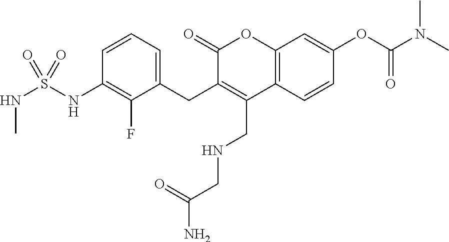 [SMILES: CNS(=O)(=O)Nc1cccc(Cc2c(CNCC(N)=O)c3ccc(OC(=O)N(C)C)cc3oc2=O)c1F] |
| 223 | 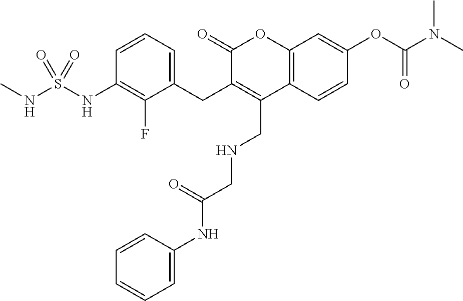 |
| 224 | 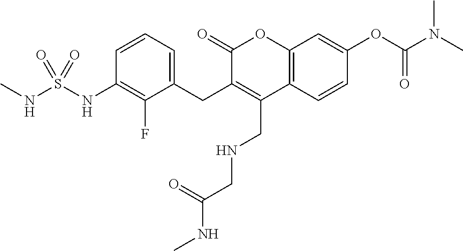 |
| 225 | 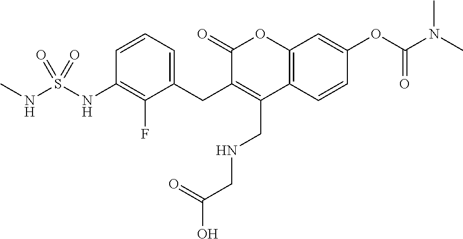 |
| 226 | 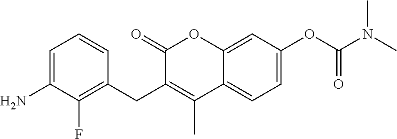 [SMILES: Cc1c(Cc2cccc(N)c2F)c(=O)oc2cc(OC(=O)N(C)C)ccc12] |
| 227 | 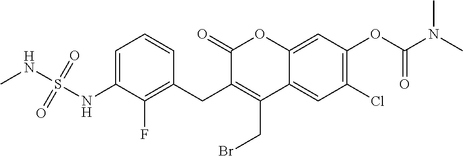 |
| 228 | 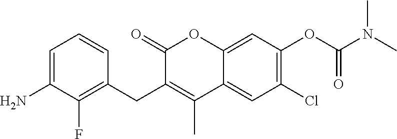 [SMILES: Cc1c(Cc2cccc(N)c2F)c(=O)oc2cc(OC(=O)N(C)C)c(Cl)cc12] |
| 229 | 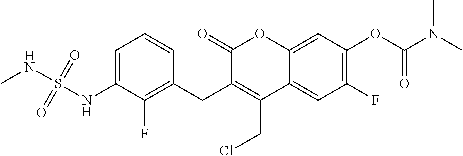 |
| 230 | 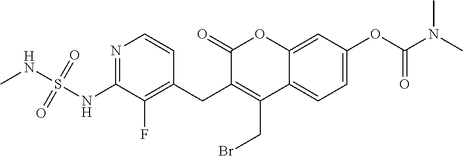 |
| 231 | 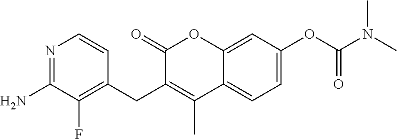 [SMILES: Cc1c(Cc2ccnc(N)c2F)c(=O)oc2cc(OC(=O)N(C)C)ccc12] |
| 232 | 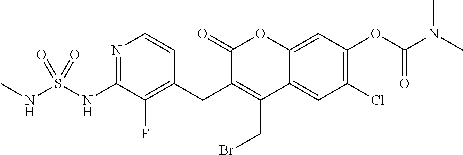 |
| 233 | 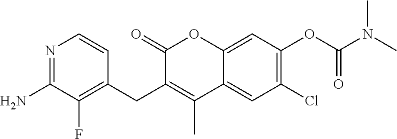 [SMILES: Cc1c(Cc2ccnc(N)c2F)c(=O)oc2cc(OC(=O)N(C)C)c(Cl)cc12] |
| 234 | 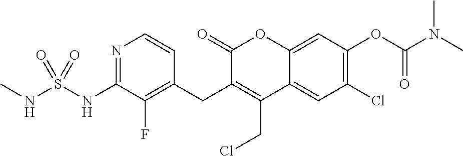 |
| 235 | 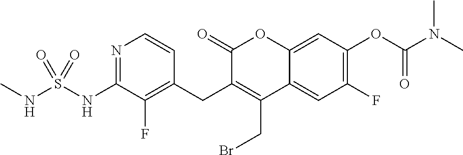 |

In some embodiments, the dual RAF/MEK inhibitor is IMM-1-104 (Immuneering) or a pharmaceutically acceptable salt thereof.

In some embodiments, the dual RAF/MEK inhibitor is TK-595 (Ikena Oncology), or a pharmaceutically acceptable salt thereof.

In some embodiments, the dual RAF/MEK inhibitor is dosed at least once a week (e.g., once a week, twice a week, three times a week, four times a week, five times a week, or six times a week). In some embodiments, the dual RAF/MEK inhibitor is dosed once a week. In some embodiments, the dual RAF/MEK inhibitor is dosed twice a week. In some embodiments, the dual RAF/MEK inhibitor is dosed three times a week.

In some embodiments, the dual RAF/MEK inhibitor is dosed at about 0.1 mg to about 100 mg, e.g., about 0.1 mg to about 50 mg, about 0.1 mg to about 10 mg, about 0.1 mg to about 5 mg, about 0.1 mg to about 4 mg, about 0.1 mg to about 3 mg, about 0.1 mg to about 2 mg, about 0.1 mg to about 1 mg, about 1 mg to about 5 mg, about 1 mg to about 10 mg, about 1 mg to about 20 mg, about 1 mg to about 40 mg, about 1 mg to about 60 mg, about 1 mg to about 80 mg, about 1 mg to about 100 mg, about 10 mg to about 100 mg, about 20 mg to about 100 mg, about 40 mg to about 100 mg, about 60 mg to about 100 mg, or about 80 mg to about 100 mg. In some embodiments, the dual RAF/MEK inhibitor is dosed at about 0.5 mg to about 10 mg per administration. In some embodiments, dual RAF/MEK inhibitor is dosed at about 0.5 mg to about 10 mg per administration. In some embodiments, dual RAF/MEK inhibitor is dosed at about 0.8 mg to about 10 mg per administration. In some embodiments, the dual RAF/MEK inhibitor is dosed at about 1 mg to about 5 mg per administration. In some embodiments, the dual RAF/MEK inhibitor is dosed at about 2 mg to about 4 mg per administration. In some embodiments, the dual RAF/MEK inhibitor is dosed at about 0.1 mg, 0.2 mg, 0.5 mg, 1 mg, 1.5 mg, 3 mg, 4 mg, 5 mg, 10 mg, 15 mg, 20 mg, 25 mg, 30 mg, 35 mg, 40 mg, 45 mg, 50 mg, 65 mg, 70 mg, 75 mg, 80 mg, 85 mg, 90 mg, 95 mg, or 100 mg per administration. In some embodiments, dual RAF/MEK inhibitor is dosed at about 4 mg per administration. In some embodiments, the dual RAF/MEK inhibitor is dosed at about 3.2 mg per administration. In some embodiments, the dual RAF/MEK inhibitor is dosed at about 2.4 mg per administration. In some embodiments, the dual RAF/MEK inhibitor is administered orally.

In some embodiments, the dual RAF/MEK inhibitor is dosed as a cycle. In some embodiments, the cycle comprises administering the dual RAF/MEK inhibitor for three weeks and then not administering the dual RAF/MEK inhibitor for one week. In some embodiments, the dual RAF/MEK inhibitor is dosed once a week. In some embodiments, the dual RAF/MEK inhibitor is dosed twice a week. In some embodiments, the dual RAF/MEK inhibitor is dosed three times a week. In some embodiments, dual RAF/MEK inhibitor is dosed at about 0.5 mg to about 10 mg per administration. In some embodiments, the dual RAF/MEK inhibitor is dosed at about 0.8 mg to about 10 mg per administration. In some embodiments, the dual RAF/MEK inhibitor is dosed at about 1 mg to about 5 mg per administration. In some embodiments, the dual RAF/MEK inhibitor is dosed at about 2 mg to about 4 mg per administration. In some embodiments, dual RAF/MEK inhibitor is dosed at about 4 mg per administration. In some embodiments, the dual RAF/MEK inhibitor is dosed at about 3.2 mg per administration. In some embodiments, the dual RAF/MEK inhibitor is dosed at about 2.4 mg per administration.

In some embodiments, the dual RAF/MEK inhibitor is dosed twice a week as a cycle, wherein the cycle comprises administering the dual RAF/MEK inhibitor for three weeks at a dose of about 0.5 mg to about 10 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of about 0.8 mg to about 10 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of about 1 mg to about 5 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of about 2 mg to about 4 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of 2.4 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of 3.2 mg per administration and then not administering the dual RAF/MEK inhibitor for one week. In some embodiments, the dual RAF/MEK inhibitor is dosed twice a week as a cycle, wherein the cycle comprises administering the dual RAF/MEK inhibitor for three weeks at a dose of 4 mg per administration and then not administering the dual RAF/MEK inhibitor for one week. In some embodiments, the cycle is repeated at least once.

In some embodiments, the dual RAF/MEK inhibitor is dosed thrice a week as a cycle, wherein the cycle comprises administering the dual RAF/MEK inhibitor for three weeks at a dose of about 0.5 mg to about 10 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of about 0.8 mg to about 10 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of about 1 mg to about 5 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of about 2 mg to about 4 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of 2.4 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of 3.2 mg per administration and then not administering the dual RAF/MEK inhibitor for one week. In some embodiments, the dual RAF/MEK inhibitor is dosed thrice a week as a cycle, wherein the cycle comprises administering the dual RAF/MEK inhibitor for three weeks at a dose of 4 mg per administration and then not administering the dual RAF/MEK inhibitor for one week. In some embodiments, the cycle is repeated at least once.

In alternative embodiments, the dual RAF/MEK inhibitor is dosed continuously (i.e., without a period of time, e.g., one week, wherein the dual RAF/MEK inhibitor is not administered). In some embodiments, the dual RAF/MEK inhibitor is dosed once a week. In some embodiments, the dual RAF/MEK inhibitor is dosed twice a week. In some embodiments, the dual RAF/MEK inhibitor is dosed three times a week.

In some embodiments, prior to administering the dual RAF/MEK inhibitor for three weeks and then not administering the dual RAF/MEK inhibitor for one week, the subject may be administered the dual RAF/MEK inhibitor for at least four weeks (i.e., without a period of time, e.g., one week, wherein the dual RAF/MEK inhibitor is not administered). In some embodiments, prior to administering the dual RAF/MEK inhibitor for three weeks and then not administering the dual RAF/MEK inhibitor for one week, the subject may be administered the dual RAF/MEK inhibitor for four weeks (i.e., without a period of time, e.g., one week, wherein the dual RAF/MEK inhibitor is not administered). In some embodiments, prior to administering the dual RAF/MEK inhibitor for three weeks and then not administering the dual RAF/MEK inhibitor for one week, the subject may be administered the dual RAF/MEK inhibitor for at eight weeks (i.e., without a period of time, e.g., two weeks, wherein the dual RAF/MEK inhibitor is not administered).

In some embodiments, prior to administering the dual RAF/MEK inhibitor twice a week for three weeks and then not administering the dual RAF/MEK inhibitor for one week, the subject may be administered the dual RAF/MEK inhibitor twice a week for at least four weeks (i.e., without a period of time, e.g., one week, wherein the dual RAF/MEK inhibitor is not administered). In some embodiments, prior to administering the dual RAF/MEK inhibitor twice a week for three weeks and then not administering the dual RAF/MEK inhibitor for one week, the subject may be administered the dual RAF/MEK inhibitor twice a week for four weeks (i.e., without a period of time, e.g., one week, wherein the dual RAF/MEK inhibitor is not administered). In some embodiments, prior to administering the dual RAF/MEK inhibitor twice a week for three weeks and then not administering the dual RAF/MEK inhibitor for one week, the subject may be administered the dual RAF/MEK inhibitor twice a week for eight weeks (i.e., without a period of time, e.g., two weeks, wherein the dual RAF/MEK inhibitor is not administered).

In some embodiments, subsequent to administering the dual RAF/MEK inhibitor for three weeks and then not administering the dual RAF/MEK inhibitor for one week, the subject may be administered the dual RAF/MEK inhibitor for at least four weeks (i.e., without a period of time, e.g., one week, wherein the dual RAF/MEK inhibitor is not administered). In some embodiments, subsequent to administering the dual RAF/MEK inhibitor for three weeks and then not administering the dual RAF/MEK inhibitor for one week, the subject may be administered the dual RAF/MEK inhibitor for four weeks (i.e., without a period of time, e.g., one week, wherein the dual RAF/MEK inhibitor is not administered). In some embodiments, subsequent to administering the dual RAF/MEK inhibitor for three weeks and then not administering the dual RAF/MEK inhibitor for one week, the subject may be administered the dual RAF/MEK inhibitor for at eight weeks (i.e., without a period of time, e.g., two weeks, wherein the dual RAF/MEK inhibitor is not administered).

In some embodiments, subsequent to administering the dual RAF/MEK inhibitor twice a week for three weeks and then not administering the dual RAF/MEK inhibitor for one week, the subject may be administered the dual RAF/MEK inhibitor twice a week for at least four weeks (i.e., without a period of time, e.g., one week, wherein the dual RAF/MEK inhibitor is not administered). In some embodiments, subsequent to administering the dual RAF/MEK inhibitor twice a week for three weeks and then not administering the dual RAF/MEK inhibitor for one week, the subject may be administered the dual RAF/MEK inhibitor twice a week for four weeks (i.e., without a period of time, e.g., one week, wherein the dual RAF/MEK inhibitor is not administered). In some embodiments, subsequent to administering the dual RAF/MEK inhibitor twice a week for three weeks and then not administering the dual RAF/MEK inhibitor for one week, the subject may be administered the dual RAF/MEK inhibitor twice a week for eight weeks (i.e., without a period of time, e.g., two weeks, wherein the dual RAF/MEK inhibitor is not administered).

### Autophagy Pathway Inhibitors

In some embodiments, the autophagy pathway inhibitor is AMPK inhibitor, L-type Ca2+ channel activator, USP10 and USP13 inhibitor, VPS34 inhibitor, or ULK inhibitor.

In some embodiments, the autophagy pathway inhibitor is chloroquine, hydroxychloroquine, apilimod, 3-methyladenine, Wortmaninn, LY294002, cycloheximide, bafilomycin A1, Lys05, leupeptin, E64d, Pepstatin A, (±)-Bay K 8644, Spautin 1, MRT 67307, MRT 68921, DCC-3116 (Deciphera Pharmaceuticals), PHY34 (Phyllanthusmin derivative for late-stage autophagy inhibition (University of Illinois)), ENV-201 (Endeavor BioMedicines), ERAS-5 (Erasca), LCC03 (5-(2′,4′-Difluorophenyl)-salicylanilide derivative, Taipei Medical University), PTU (1-phenyl 2-thiourea, Hong Kong Polytechnic University), siroquine (JP001, HebaBiz Biotech), SB02024 (Deciphera Pharmaceuticals), GCT-VPS34 (Global Cancer Technology), Vescor01 (Vescor Therapeutics), S-130 (Sun Yat-sen University), SBI-0206965 (Salk Institute for Biological Studies), SMER-28 (Harvard Medical School), FMK-9A (F. Hoffmann-La Roche Ltd), small molecule to inhibit ULK1 for solid tumor (TXINNO Bioscience Inc), or small molecule to inhibit UNC-51-like kinase 1 (Scripps Research Institute).

In some embodiments, the autophagy pathway inhibitor is LY294002, having the following structure:

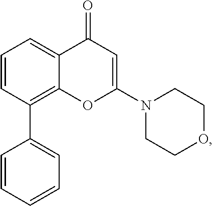 [SMILES: O=c1cc(N2CCOCC2)oc2c(-c3ccccc3)cccc12]

or a pharmaceutically acceptable salt thereof.

In some embodiments, the autophagy pathway inhibitor is MRT 67307, having the following structure:

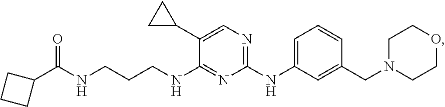 [SMILES: O=C(NCCCNc1nc(Nc2cccc(CN3CCOCC3)c2)ncc1C1CC1)C1CCC1]

or a pharmaceutically acceptable salt thereof.

In some embodiments, the autophagy pathway inhibitor is MRT 68921, having the following structure:

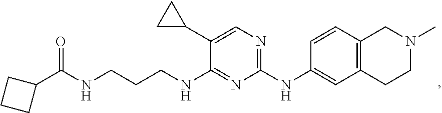 [SMILES: CN1CCc2cc(Nc3ncc(C4CC4)c(NCCCNC(=O)C4CCC4)n3)ccc2C1]

or a pharmaceutically acceptable salt thereof.

In some embodiments, the autophagy pathway inhibitor is SBI-0206965, having the following structure:

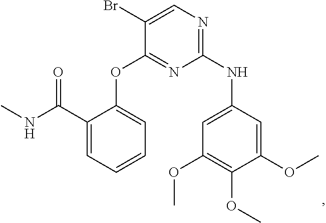

or a pharmaceutically acceptable salt thereof.

In some embodiments, the autophagy pathway inhibitor is NSC-185058, having the following structure:

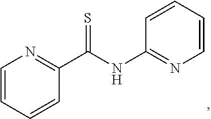 [SMILES: S=C(Nc1ccccn1)c1ccccn1]

or a pharmaceutically acceptable salt thereof.

In some embodiments, the autophagy pathway inhibitor is dosed at about 0.1 mg to about 5000 mg, e.g., about 1 mg to about 3000 mg, about 10 mg to about 2000 mg, e.g., about 100 mg to about 2000 mg, about 100 mg to about 1500 mg, about 100 mg to about 1000 mg, about 100 mg to about 800 mg, about 100 mg to about 600 mg, about 100 mg to about 400 mg, about 100 mg to about 200 mg, about 200 mg to about 2000 mg, about 200 mg to about 1500 mg, about 200 mg to about 1000 mg, about 200 mg to about 800 mg, about 200 mg to about 600 mg, about 200 mg to about 400 mg, about 400 mg to about 2000 mg, about 400 mg to about 1500 mg, about 400 mg to about 1000 mg, about 400 mg to about 800 mg, about 400 mg to about 600 mg, about 600 mg to about 2000 mg, about 600 mg to about 1500 mg, about 600 mg to about 1000 mg, about 600 mg to about 800 mg, about 800 mg to about 2000 mg, 800 mg to about 1500 mg, about 800 mg to about 1000 mg, about 600 mg to about 2000 mg, about 600 mg to about 1500 mg, about 600 mg to about 1000 mg, about 600 mg to about 800 mg per administration. In some embodiments, the autophagy pathway inhibitor is dosed at 10 mg to 2000 mg per administration. In some embodiments, the autophagy pathway inhibitor is dosed at 100 mg to 1000 mg per administration. In some embodiments, the autophagy pathway inhibitor is dosed at 100 mg to 200 mg per administration. In some embodiments, the autophagy pathway inhibitor is dosed at 300 mg to 500 mg per administration. In some embodiments, the autophagy pathway inhibitor is dosed at 200 mg to 600 mg per administration. In some embodiments, the autophagy pathway inhibitor is dosed at 800 mg to 1000 mg per administration. In some embodiments, the autophagy pathway inhibitor is dosed at about 1 mg per administration. In some embodiments, the autophagy pathway inhibitor is dosed at about 5 mg per administration. In some embodiments, the autophagy pathway inhibitor is dosed at about 10 mg per administration. In some embodiments, the autophagy pathway inhibitor is dosed at about 50 mg per administration. In some embodiments, the autophagy pathway inhibitor is dosed at about 100 mg per administration. In some embodiments, the autophagy pathway inhibitor is dosed at about 150 mg per administration. In some embodiments, the autophagy pathway inhibitor is dosed at about 200 mg per administration. In some embodiments, the autophagy pathway inhibitor is dosed at about 250 mg per administration. In some embodiments, the autophagy pathway inhibitor is dosed at about 300 mg per administration. In some embodiments, the autophagy pathway inhibitor is dosed at about 350 mg per administration. In some embodiments, the autophagy pathway inhibitor is dosed at about 400 mg per administration. In some embodiments, the autophagy pathway inhibitor is dosed at about 450 mg per administration. In some embodiments, the autophagy pathway inhibitor is dosed at about 500 mg per administration. In some embodiments, the autophagy pathway inhibitor is dosed at about 550 mg per administration. In some embodiments, the autophagy pathway inhibitor is dosed at about 600 mg per administration. In some embodiments, the autophagy pathway inhibitor is dosed at about 650 mg per administration. In some embodiments, the autophagy pathway inhibitor is dosed at about 700 mg per administration. In some embodiments, the autophagy pathway inhibitor is dosed at about 750 mg per administration. In some embodiments, the autophagy pathway inhibitor is dosed at about 800 mg per administration. In some embodiments, the autophagy pathway inhibitor is dosed at about 850 mg per administration. In some embodiments, the autophagy pathway inhibitor is dosed at about 900 mg per administration. In some embodiments, the autophagy pathway inhibitor is dosed at about 950 mg per administration. In some embodiments, the autophagy pathway inhibitor is dosed at about 1000 mg per administration.

In some embodiments, the autophagy pathway inhibitor is administered at least once a week. In some embodiments, the autophagy pathway inhibitor is administered once a week. In some embodiments, the autophagy pathway inhibitor is administered twice a week. In some embodiments, the autophagy pathway inhibitor is administered thrice a week. In some embodiments, the autophagy pathway inhibitor is administered four times a week. In some embodiments, the autophagy pathway inhibitor is administered five times a week. In some embodiments, the autophagy pathway inhibitor is administered six times a week. In some embodiments, the autophagy pathway inhibitor is administered at least once daily. In some embodiments, the autophagy pathway inhibitor is administered once daily. In some embodiments, the autophagy pathway inhibitor is administered twice daily. In some embodiments, the autophagy pathway inhibitor is administered orally.

In some embodiments, the dual RAF/MEK inhibitor is administered before the autophagy pathway inhibitor is administered. In some embodiments, the dual RAF/MEK inhibitor is administered after the autophagy pathway inhibitor is administered. In some embodiments, the dual RAF/MEK inhibitor is administered concurrently with the autophagy pathway inhibitor.

In some embodiments, the dual RAF/MEK inhibitor and the autophagy pathway inhibitor are independently dosed cyclically.

In some embodiments, the dual RAF/MEK inhibitor and the autophagy pathway inhibitor are simultaneously dosed cyclically.

### FAK Inhibitors

Potent inhibitors of the FAK protein tyrosine kinases may be adapted to therapeutic use as antiproliferative agents (e.g., anticancer), antitumor (e.g., effective against solid tumors), antiangiogenesis (e.g., stop or prevent proliferation of blood vessels) in mammals, particularly in humans. In some embodiments, the methods described herein further contemplate administering to the subject a FAK inhibitor described herein. The FAK inhibitors may be useful in the prevention and treatment of non-hematologic malignancies, a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH), and in the prevention and treatment of disorders such as mesothelioma. In some embodiments, the compounds described herein, e.g., FAK inhibitors, inhibit protein tyrosine kinase 2 (PYK2).

In some embodiments, the methods described herein further contemplate administering to the subject an effective amount of a FAK inhibitor.

An exemplary FAK inhibitor includes, but is not limited to, defactinib having the following structure:

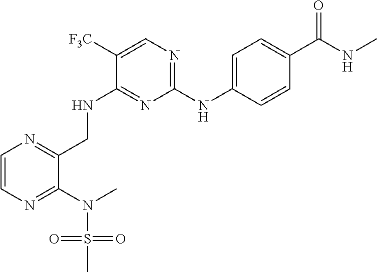 [SMILES: CNC(=O)c1ccc(Nc2ncc(C(F)(F)F)c(NCc3nccnc3N(C)S(C)(=O)=O)n2)cc1]

or a pharmaceutically acceptable salt thereof. Defactinib is also known as VS-6063 (e.g., VS-6063 free base) or PF-04554878. VS-6063 and related compounds are also disclosed in, for example, U.S. Pat. No. 7,928,109, the content of which is incorporated herein by reference. In some embodiments, VS-6063 can form a pharmaceutically acceptable salt (e.g., VS-6063 hydrochloride).

In some embodiments, the FAK inhibitor is VS-4718, having the following structure:

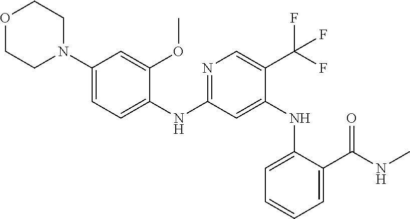 [SMILES: CNC(=O)c1ccccc1Nc1cc(Nc2ccc(N3CCOCC3)cc2OC)ncc1C(F)(F)F]

or a pharmaceutically acceptable salt thereof.

In some embodiments, the FAK inhibitor is TAE226, having the following structure:

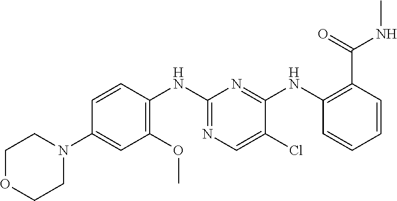 [SMILES: CNC(=O)c1ccccc1Nc1nc(Nc2ccc(N3CCOCC3)cc2OC)ncc1Cl]

or a pharmaceutically acceptable salt thereof.

In some embodiments, the FAK inhibitor is GSK2256098, having the following structure:

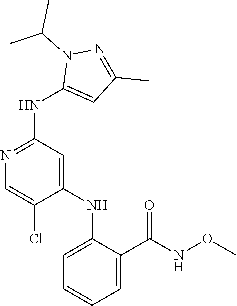

or a pharmaceutically acceptable salt thereof.

In some embodiments, the FAK inhibitor is PF-03814735, having the following structure:

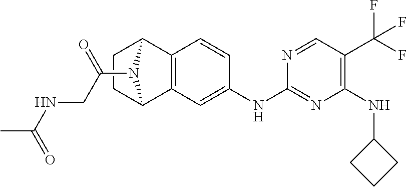 [SMILES: CC(=O)NCC(=O)N1[C@@H]2CC[C@H]1c1ccc(Nc3ncc(C(F)(F)F)c(NC4CCC4)n3)cc12]

or a pharmaceutically acceptable salt thereof.

In some embodiments, the FAK inhibitor is BI-4464, having the following structure:

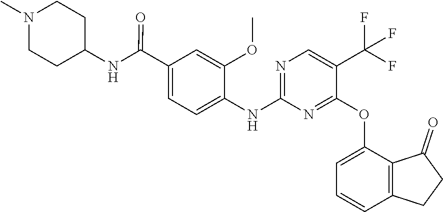 [SMILES: COc1cc(C(=O)NC2CCN(C)CC2)ccc1Nc1ncc(C(F)(F)F)c(Oc2cccc3c2C(=O)CC3)n1]

or a pharmaceutically acceptable salt thereof.

In some embodiments, the FAK inhibitor is BI-853520 (IN10018; Boehringer Ingelheim). In some other embodiments, the FAK inhibitor is APG-2449 (Ascentage Pharma Group).

In some embodiments, the FAK inhibitor is selected from the group consisting of defactinib, TAE226, BI-853520, GSK2256098, PF-03814735, BI-4464, VS-4718, and APG-2449, or a pharmaceutically acceptable salt thereof. For example, the FAK inhibitor is defactinib or a pharmaceutically acceptable salt thereof.

In some embodiments, the FAK inhibitor (e.g., defactinib) is dosed at least once daily. For example, in some embodiments, the FAK inhibitor (e.g., defactinib) is dosed once daily. In some embodiments, the FAK inhibitor (e.g., defactinib) is dosed twice daily.

In some embodiments, the FAK inhibitor (e.g., defactinib) is dosed at about 100 mg to about 1000 mg, e.g., about 100 mg to about 800 mg, about 100 mg to about 600 mg, about 100 mg to about 400 mg, about 100 mg to about 200 mg, about 200 mg to about 1000 mg, about 400 mg to about 1000 mg, about 600 mg to about 1000 mg, about 800 mg to about 1000 mg, about 200 mg to about 800 mg, about 200 mg to about 600 mg, about 200 mg to about 400 mg, about 400 mg to about 800 mg, or about 400 mg to about 600 mg per administration. In some embodiments, the FAK inhibitor (e.g., defactinib) is dosed at about 200 mg to about 400 mg per administration. In some embodiments, the FAK inhibitor (e.g., defactinib) is dosed at about 100 mg per administration. In some embodiments, the FAK inhibitor (e.g., defactinib) is dosed at about 200 mg per administration. In some embodiments, the FAK inhibitor (e.g., defactinib) is dosed at about 300 mg per administration. In some embodiments, the FAK inhibitor (e.g., defactinib) is dosed at about 400 mg per administration. In some embodiments, the FAK inhibitor (e.g., defactinib) is dosed at about 500 mg per administration. In some embodiments, the FAK inhibitor (e.g., defactinib) is dosed at about 600 mg per administration. In some embodiments, the FAK inhibitor (e.g., defactinib) is administered orally.

In some embodiments, the FAK inhibitor is dosed as a cycle, wherein the cycle comprises administering the FAK inhibitor for three weeks and then not administering the FAK inhibitor for one week. In some embodiments, the cycle is repeated at least once.

In some embodiments, the dual RAF/MEK inhibitor and the FAK inhibitor (e.g., defactinib, or a pharmaceutically acceptable salt thereof) are independently dosed cyclically.

In some embodiments, the dual RAF/MEK inhibitor and the FAK inhibitor (e.g., defactinib, or a pharmaceutically acceptable salt thereof) are simultaneously dosed cyclically.

In some embodiments, prior to administering the FAK inhibitor for three weeks and then not administering the FAK inhibitor for one week, the subject may be administered the FAK inhibitor for at least four weeks (i.e., without a period of time, e.g., one week, wherein the FAK inhibitor is not administered). In some embodiments, prior to administering the FAK inhibitor for three weeks and then not administering the FAK inhibitor for one week, the subject may be administered the FAK inhibitor for four weeks (i.e., without a period of time, e.g., one week, wherein the FAK inhibitor is not administered). In some embodiments, prior to administering the FAK inhibitor for three weeks and then not administering the FAK inhibitor for one week, the subject may be administered the FAK inhibitor for at eight weeks (i.e., without a period of time, e.g., two weeks, wherein the FAK inhibitor is not administered).

In some embodiments, prior to administering the FAK inhibitor once or twice a day for three weeks and then not administering the FAK inhibitor for one week, the subject may be administered the FAK inhibitor once or twice a day for at least four weeks (i.e., without a period of time, e.g., one week, wherein the FAK inhibitor is not administered). In some embodiments, prior to administering the FAK inhibitor once or twice a day for three weeks and then not administering the FAK inhibitor for one week, the subject may be administered the FAK inhibitor once or twice a day for four weeks (i.e., without a period of time, e.g., one week, wherein the FAK inhibitor is not administered). In some embodiments, prior to administering the FAK inhibitor once or twice a day for three weeks and then not administering the FAK inhibitor for one week, the subject may be administered the FAK inhibitor once or twice a day for eight weeks (i.e., without a period of time, e.g., two weeks, wherein the FAK inhibitor is not administered).

In some embodiments, subsequent to administering the FAK inhibitor for three weeks and then not administering the FAK inhibitor for one week, the subject may be administered the FAK inhibitor for at least four weeks (i.e., without a period of time, e.g., one week, wherein the FAK inhibitor is not administered). In some embodiments, subsequent to administering the FAK inhibitor for three weeks and then not administering the FAK inhibitor for one week, the subject may be administered the FAK inhibitor for four weeks (i.e., without a period of time, e.g., one week, wherein the FAK inhibitor is not administered). In some embodiments, subsequent to administering the FAK inhibitor for three weeks and then not administering the FAK inhibitor for one week, the subject may be administered the FAK inhibitor for at eight weeks (i.e., without a period of time, e.g., two weeks, wherein the FAK inhibitor is not administered).

In some embodiments, subsequent to administering the FAK inhibitor once or twice a day for three weeks and then not administering the FAK inhibitor for one week, the subject may be administered the FAK inhibitor once or twice a day for at least four weeks (i.e., without a period of time, e.g., one week, wherein the FAK inhibitor is not administered). In some embodiments, subsequent to administering the FAK inhibitor once or twice a day for three weeks and then not administering the FAK inhibitor for one week, the subject may be administered the FAK inhibitor once or twice a day for four weeks (i.e., without a period of time, e.g., one week, wherein the FAK inhibitor is not administered). In some embodiments, subsequent to administering the FAK inhibitor once or twice a day for three weeks and then not administering the FAK inhibitor for one week, the subject may be administered the FAK inhibitor once or twice a day for eight weeks (i.e., without a period of time, e.g., two weeks, wherein the FAK inhibitor is not administered).

### Diseases and Disorders

### Abnormal Cell Growth

Abnormal cell growth, as used herein and unless otherwise indicated, refers to cell growth that is independent of normal regulatory mechanisms (e.g., loss of contact inhibition). This includes the abnormal growth of: (1) tumor cells (tumors) that proliferate, for example, by expressing a mutated tyrosine kinase or overexpression of a receptor tyrosine kinase; (2) benign and malignant cells of other proliferative diseases, for example, in which aberrant tyrosine kinase activation occurs; (3) any tumors that proliferate, for example, by receptor tyrosine kinases; (4) any tumors mat proliferate, for example, by aberrant serine/threonine kinase activation; and (5) benign and malignant cells of other proliferative diseases, for example, in which aberrant serine/threonine kinase activation occurs. Abnormal cell growth can refer to cell growth in epithelial (e.g., carcinomas, adenocarcinomas): mesenchymal (e.g., sarcomas (e.g. leiomyosarcoma. Ewing's sarcoma)); hematopoetic (e.g., lymphomas, leukemias, myelodysplasias (e.g., pre-malignant)); or other (e.g., melanoma, mesothelioma, and other tumors of unknown origin) cell.

### Neoplastic Disorders

Abnormal cell growth can refer to a neoplastic disorder. A “neoplastic disorder” is a disease or disorder characterized by cells that have the capacity for autonomous growth or replication, e.g., an abnormal state or condition characterized by proliferative cell growth. An abnormal mass of tissue as a result of abnormal cell growth or division, or a “neoplasm,” can be benign, pre-malignant (carcinoma in situ) or malignant (cancer).

Exemplary neoplastic disorders include: carcinoma, sarcoma, metastatic disorders (e.g., tumors arising from prostate, colon, lung, breast and liver origin), hematopoietic neoplastic disorders, e.g., leukemias, metastatic tumors. Treatment with the compound may be in an amount effective to ameliorate at least one symptom of the neoplastic disorder, e.g., reduced cell proliferation, reduced tumor mass, etc.

### Cancer

The inventive methods of the present invention may be useful in the prevention and treatment of cancer, including for example, solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer (e.g., Hepatocellular carcinoma), non-small cell carcinoma of the lung, pancreatic (e.g., metastatic pancreatic adenocarcinoma) and cancer of the small intestine.

The cancer can include mesothelioma; neurofibromatosis; e.g., neurofibromatosis type 2, neurofibromatosis type 1; renal cancer; lung cancer, non small cell lung cancer; liver cancer; thyroid cancer; ovarian; breast cancer; a nervous system tumor; schwannoma; meningioma; schwannomatosis; neuroma acoustic; adenoid cystic carcinoma; ependymoma; ependymal tumors, or any other tumor which exhibits decreased merlin expression and/or mutation, and/or deletion and/or promotor hypermethylation of the NF-2 gene. In some embodiments, the cancer is renal cancer.

For example, the cancer may include, but is not limited to, ovarian cancer, non-small cell lung cancer (e.g., NSCLC adenocarcinoma)), uterine endometrioid carcinoma, pancreatic adenocarcinoma, colorectal adenocarcinoma, colorectal cancer, pancreatic cancer, or lung adenocarcinoma. In some embodiments, the NSCLC is characterized as having a KRAS mutation. In some embodiments, the ovarian cancer is low grade serous ovarian cancer.

The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells (e.g., a tumor enriched with cells that have undergone an epithelial-to-mesenchymal transition or a metastatic tumor).

The cancer can include pancreatic cancer, gynecologic cancer (e.g., cervical cancer, ovarian cancer, uterine cancer, vaginal cancer, endometrial cancer, or vulvar cancer), liver cancer, prostate cancer, mesothelioma, breast cancer, bladder cancer, melanoma, lung cancer, colorectal cancer, thyroid cancer, glioblastoma, or renal cancer. In some embodiments, the cancer is melanoma, lung cancer, colorectal cancer, thyroid cancer, glioblastoma, or renal cancer. In some embodiments, the lung cancer is non-small cell lung cancer. In some embodiments, the lung cancer is metastatic non-small cell lung cancer. In some embodiments, the melanoma is unresectable melanoma. In some embodiments, the melanoma is metastatic melanoma. In some embodiments, the cancer is colorectal cancer. In some embodiments, the thyroid cancer is papillary thyroid cancer. In some embodiments, the thyroid cancer is follicular thyroid cancer. In some embodiments, the thyroid cancer is anaplastic thyroid cancer.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

The cancer can also include for example, but is not limited to, epithelial cancers, breast, lung, pancreatic, colorectal (e.g., metastatic colorectal, e.g., metastatic KRAS mutated), prostate, head and neck, melanoma (e.g., NRAS mutated locally advanced or metastatic malignant cutaneous melanoma), acute myelogenous leukemia, and glioblastoma. Exemplary breast cancers include triple negative breast cancer, basal-like breast cancer, claudin-low breast cancer, invasive, inflammatory, metaplastic, and advanced HER-2 positive or ER-positive cancers resistant to therapy.

In some embodiments, the cancer is characterized as having a RAS mutation. In some embodiments, the cancer is a cancer characterized as having a KRAS mutation. In some embodiments, the cancer is a cancer characterized as having a NRAS mutation. In some embodiments, the cancer is a cancer characterized as having a HRAS mutation.

In some embodiments, the cancer is a cancer characterized as having a RAF mutation. In some embodiments, the cancer is a cancer characterized as having a BRAF mutation.

The cancer can also include lung adenocarcinoma, colorectal cancer (CRC), uveal melanoma, ovarian cancer, uterine endometrioid carcinoma, bladder urothelial carcinoma, breast invasive lobular carcinoma, cervical squamous cell carcinoma, cutaneous melanoma, endocervical adenocarcinoma, hepatocellular carcinoma, pancreatic adenocarcinoma, biphasic type pleural mesothelioma, renal clear cell carcinoma, renal clear cell carcinoma, stomach adenocarcinoma, tubular stomach adenocarcinoma, uterine carcinosarcoma, or uterine malignant mixed Mullerian tumor.

In some embodiments, the cancer is unresectable or metastatic melanoma, melanoma with lymph node involvement or metastatic disease who have undergone complete resection, metastatic non-small cell lung cancer and progression on or after platinum-based chemotherapy, metastatic small cell lung cancer with progression after platinum-based chemotherapy and at least one other line of therapy, advanced renal cell carcinoma who have received prior antiangiogenic therapy, advanced renal cell carcinoma, classical Hodgkin lymphoma, recurrent or metastatic squamous cell carcinoma of the head and neck with disease progression on or after a platinum-based therapy, locally advanced or metastatic urothelial carcinoma, microsatellite instability-high (MSI-H) or mismatch repair deficient (dMMR) metastatic colorectal cancer, or hepatocellular carcinoma.

In some embodiments, the cancer is melanoma, non-small cell lung cancer, small cell lung cancer, head and neck squamous cell cancer, classical Hodgkin lymphoma, primary mediastinal large B-cell lymphoma, urothelial carcinoma, microsatellite instability-high cancer, gastric cancer, esophageal cancer, cervical cancer, hepatocellular carcinoma, merkel cell carcinoma, renal cell carcinoma, or endometrial carcinoma.

Other cancers include but are not limited to, uveal melanoma, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood and bone marrow cancers (e.g., advanced hematological malignancies, leukemia, e.g., acute myeloid leukemia (e.g., primary or secondary), acute lymphoblastic leukemia, acute lymphocytic leukemia, T cell leukemia, hematological malignancies, advanced myeloproliferative disorders, myelodysplastic syndrome, relapsed or refractory multiple myeloma, advanced myeloproliferative disorders), retinal, bladder, cervical, kidney, endometrial, meningioma, lymphoma, skin, uterine, lung, non small cell lung, nasopharyngeal carcinoma, neuroblastoma, solid tumor, hematologic malignancy, squamous cell carcinoma, testicular, thyroid, mesothelioma, brain vulval, sarcoma, intestine, oral, endocrine, salivary, spermatocyte seminoma, sporadic medulalry thyroid carcinoma, non-proliferating testes cells, cancers related to malignant mast cells, non-Hodgkin's lymphoma, and diffuse large B cell lymphoma.

In some embodiments, the tumor is a solid tumor. In some embodiments, the solid tumor is locally advanced or metastatic, hi some embodiments, the solid tumor is refractory (e.g., resistant) after standard therapy.

Methods described herein can reduce, ameliorate or altogether eliminate the disorder, and/or its associated symptoms, to keep it from becoming worse, to slow the rate of progression, or to minimize the rate of recurrence of the disorder once it has been initially eliminated (i.e., to avoid a relapse). A suitable dose and therapeutic regimen may vary depending upon the specific compounds, combinations, and/or pharmaceutical compositions used and the mode of delivery of the compounds, combinations, and/or pharmaceutical compositions. In some embodiments, the method increases the average length of survival, increases the average length of progression-free survival, and/or reduces the rate of recurrence, of subjects treated with the combinations described herein in a statistically significant manner.

In some embodiments, the cancer is lung cancer (e.g., non-small cell lung cancer CNSCLC), e.g., KRAS mutant NSCLC; metastatic cancer), bone cancer, pancreatic cancer, skin cancer, cancer of the head or neck, cutaneous or intraocular melanoma, uterine cancer, ovarian cancer (e.g., unresectable low-grade ovarian, advanced or metastatic ovarian cancer), rectal cancer, cancer of the anal region, stomach cancer, colon cancer, breast cancer (e.g., triple-negative breast cancer (e.g., breast cancer which does not express the genes for the estrogen receptor, progesterone receiptor, and Her2/neu)), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, mesothelioma (e.g., malignant pleural mesothelioma, e.g., surgical resectable malignant pleural mesothelioma) or a combination of one or more of the foregoing cancers. In some embodiments, the cancer is metastatic. In some embodiments, the abnormal cell growth is locally recurring (e.g., the subject has a locally recurrent disease, e.g., cancer).

### Additional Therapies

In some embodiments, the methods and compositions described herein is administered together with an additional therapy or additional agent. In one embodiment, a mixture of one or more compounds or pharmaceutical compositions may be administered with the combination described herein to a subject in need thereof. In yet another embodiment, one or more compounds or compositions (e.g., pharmaceutical compositions) may be administered with the combination described herein for the treatment or avoidance of various diseases, including, for example, cancer, diabetes, neurodegenerative diseases, cardiovascular disease, blood clotting, inflammation, flushing, obesity, aging, stress, etc. In various embodiments, combination therapies comprising a compound or pharmaceutical composition described herein may refer to (1) pharmaceutical compositions that comprise one or more compounds in combination with the combination described herein; and (2) co-administration of one or more compounds or pharmaceutical compositions described herein with the combination described herein, wherein the compound or pharmaceutical composition described herein have not been formulated in the same compositions. In some embodiments, the combinations described herein is administered with an additional treatment (e.g., an additional cancer treatment). In some embodiments, the additional treatment (e.g., an additional cancer treatment) can be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment (e.g., a compound or therapy). The order of administration of the first and secondary compound or therapy can also be reversed.

Exemplary cancer treatments include, for example: chemotherapy, targeted therapies such as antibody therapies, immunotherapy, and hormonal therapy. Examples of each of these treatments are provided below.

### Chemotherapy

In some embodiments, a combination described herein is administered with a chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptotnecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixanlrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfm, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time or sequentially. Often, two or more chemotherapy agents are used as combination chemotherapy. In some embodiments, the chemotherapy agents (including combination chemotherapy) can be used in combination with a combination described herein.

### Targeted Therapy

In some embodiments, a combination described herein is administered with a targeted therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, desatinib, erolotinib, imatinib, gefitinib, lapatinib, Lestaurtinib, Nilotinib, Semaxanib, Sorafenib, Sunitinib, and Vandetanib, and also cyclin-depdendent kinase inhibitors such as Alvocidib and Seliciclib. Monoclonal antibody therapy is another strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include Ctuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a combination described herein.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decay s in the vicinity of the cell. An example of such therapy includes BEXXAR®.

### Immunotherapy

In some embodiments, a combination described herein is administered with an immunotherapy. Cancer immunotherapy refers to a diverse set of therapeutic strategies designed to induce the subject's own immune system to fight the tumor.

Contemporary methods for generating an immune response against tumors include intravesicular BCG immunotherapy for superficial bladder cancer, and use of interferons and other cytokines to induce an immune response in subjects with renal cell carcinoma and melanoma. Allogeneic hematopoietic stem cell transplantation can be considered a form of immunotherapy, since the donor's immune cells will often attack the tumor in a graft-versus-tumor effect. In some embodiments, the immunotherapy agents can be used in combination with a combination as described herein.

### Hormonal Therapy

In some embodiments, a combination described is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a combination described herein.

In some embodiments, the additional agent is an agent that modifies ER, PR, and/or AR. For example, the additional agent is an AR antagonist, which includes, but is not limited to, flutamide, bicalutamide and nilutamide. In some embodiments, the additional agent is an agent that blocks estrogen or progesterone, which includes, aromatase inhibitors including but is not limited to, anastrozole, letrozole, and exemestane. In some embodiments, the additional agent is an estrogen receptor modulator including, but not limited to, fulvetrant, tamoxifen and raloxifene.

### Radiation Therapy

The combinations described herein can be used in combination with directed energy or particle, or radioisotope treatments, e.g., radiation therapies, e.g., radiation oncology, for the treatment of proliferative disease, e.g., cancer, e.g., cancer associated with cancer stem cells. The combinations described herein may be administered to a subject simultaneously or sequentially along with the directed energy or particle, or radioisotope treatments. For example, the combinations described herein may be administered before, during, or after the directed energy or particle, or radioisotope treatment, or a combination thereof. The directed energy or particle therapy may comprise total body irradiation, local body irradiation, or point irradiation. The directed energy or particle may originate from an accelerator, synchrotron, nuclear reaction, vacuum tube, laser, or from a radioisotope. The therapy may comprise external beam radiation therapy, teletherapy, brachy therapy, sealed source radiation therapy, systemic radioisotope therapy, or unsealed source radiotherapy. The therapy may comprise ingestion of, or placement in proximity to, a radioisotope, e.g., radioactive iodine, cobalt, cesium, potassium, bromine, fluorine, carbon. External beam radiation may comprise exposure to directed alpha particles, electrons (e.g., beta particles), protons, neutrons, positrons, or photons (e.g., radiowave, millimeter wave, microwave, infrared, visible, ultraviolet, X-ray, or gamma-ray photons). The radiation may be directed at any portion of the subject in need of treatment.

### Surgery

The combinations described herein can be used in combination with surgery, e.g., surgical exploration, intervention, biopsy, for the treatment of proliferative disease, e.g., cancer, e.g., cancer associated with cancer stem cells. The combinations described herein may be administered to a subject simultaneously or sequentially along with the surgery. For example, the combinations described herein may be administered before (preoperative), during, or after (post-operative) the surgery, or a combination thereof. The surgery may be a biopsy during which one or more cells are collected for further analysis. The biopsy may be accomplished, for example, with a scalpel, a needle, a catheter, an endoscope, a spatula, or scissors. The biopsy may be an excisional biopsy, an incisional biopsy, a core biopsy, or a needle biopsy, e.g., a needle aspiration biopsy. The surgery may involve the removal of localized tissues suspected to be or identified as being cancerous. For example, the procedure may involve the removal of a cancerous lesion, lump, polyp, or mole. The procedure may involve the removal of larger amounts of tissue, such as breast, bone, skin, fat, or muscle. The procedure may involve removal of part of, or the entirety of, an organ or node, for example, lung, throat, tongue, bladder, cervix, ovary, testicle, lymph node, liver, pancreas, brain, eye, kidney, gallbladder, stomach, colon, rectum, or intestine. In one embodiment, the cancer is breast cancer, e.g., triple negative breast cancer, and the surgery is a mastectomy or lumpectomy.

### Anti-Inflammatory Agents

A combination described herein can be administered with an anti-inflammatory agent. Anti-inflammatory agents can include, but are not limited to, non-steroidal anti-inflammatory agents (e.g., Salicylates (Aspirin (acetylsalicylic acid), Diflunisal, Salsalate), Propionic acid derivatives (Ibuprofen, Naproxen, Fenoprofen, Ketoprofen, Flurbiprofen, Oxaprozin, Loxoprofen), Acetic acid derivatives (Indomethacin, Sulindac, Etodolac, Ketorolac, Diclofenac, Nabumetone), Enolic acid (Oxicam) derivatives (Piroxicam, Meloxicam, Tenoxicam, Droxicam, Lomoxicam, Isoxicam), Fenamic acid derivatives (Fenamates) (Mefenamic acid, Meclofenamic acid, Flufenamic acid. Tolfenamic acid). Selective COX-2 inhibitors (Coxibs) (Ceiecoxib), Sulphonanilides (Nimesulide). Steriods (e.g. Hydrocortisone (Cortisol), Cortisone acetate, Prednisone, Prednisolone, Methylprednisolone, Dexamethasone, Betamethasone, Triamcinolone, Beclometasone, Fludrocortisone acetate, Deoxycorticosterone acetate, Aldosterone).

### Analgesic Agents

Analgesics can include but are not limited to, opiates (e.g. morphine, codeine, oxycodone, hydrocodone, dihydromorphine, pethidine, buprenorphine, tramadol, venlafaxine), paracetomal and Nonsteroidal anti-inflammatory agents (e.g., Salicylates (Aspirin (acetylsalicylic acid), Diflunisal, Salsalate), Propionic acid derivatives (Ibuprofen, Naproxen, Fenoprofen, Ketoprofen, Flurbiprofen, Oxaprozin, Loxoprofen), Acetic acid derivatives (Indomethacin, Sulindac, Etodolac, Ketorolac, Diclofenac, Nabumetone), Enolic acid (Oxicam) derivatives (Piroxicam, Meloxicam, Tenoxicam, Droxicam, Lomoxicam, Isoxicam), Fenamic acid derivatives (Fenamates)(Mefenamic acid, Meclofenamic acid, Flufenamic acid. Tolfenamic acid). Selective COX-2 inhibitors (Coxibs) (Ceiecoxib), Sulphonanilides (Nimesulide).

### Antiemetic Agents

A combination described herein can be administered with an antiemetic agent. Antiemetic agents can include, but are not limited to, 5-HT3 receptor antagonists (Dolasetron (Anzemet), Granisetron (Kytril, Sancuso), Ondansetron (Zofran), Tropisetron (Navoban), Palonosetron (Aloxi), Mirtazapine (Remeron)), Dopamine antagonists (Domperidone, Olanzapine, Droperidol, Haloperidol, Chlorpromazine, Promethazine, Prochlorperazine, Metoclopramide (Reglan), Alizapride, Prochlorperazine (Compazine, Stemzine, Buccastem, Stemetil, Phenotil), NKl receptor antagonist (Aprepitant (Emend), Antihistamines (Cyclizine, Diphenhydramine (Benadryl), Dimenhydrinate (Gravol, Dramamine), Meclozine (Bonine, Antivert), Promethazine (Pentazine, Phenergan, Promacot), Hydroxyzine), benzodiazapines (Lorazepam, Midazolam), Anticholinergics (hyoscine), steroids (Dexamethasone).

### Combinations

The phrase, “in combination with,” and the terms “co-administration,” “co-administering,” or “co-providing”, as used herein in the context of the administration of a compound described herein or a therapy described herein, means that two (or more) different compounds or therapies are delivered to the subject during the course of the subject's affliction with the disease or disorder (e.g., a disease or disorder as described herein, e.g., cancer), e.g., two (or more) different compounds or therapies are delivered to the subject after the subject has been diagnosed with the disease or disorder (e.g., a disease or disorder as described herein, e.g., cancer) and before the disease or disorder has been cured or eliminated or treatment has ceased for other reasons.

In some embodiments, the delivery of one compound or therapy is still occurring when the delivery of the second begins, so that there is overlap in terms of administration. This is sometimes referred to herein as “simultaneous” or “concurrent delivery.” In other embodiments, the delivery of one compound or therapy ends before the delivery of the other compound or therapy begins. In some embodiments of either case, the treatment (e.g., administration of compound, composition, or therapy) is more effective because of combined administration. For example, the second compound or therapy is more effective, e.g., an equivalent effect is seen with less of the second compound or therapy, or the second compound or therapy reduces symptoms to a greater extent, than would be seen if the second compound or therapy were administered in the absence of the first compound or therapy, or the analogous situation is seen with the first compound or therapy. In some embodiments, delivery is such that the reduction in a symptom, or other parameter related to the disorder is greater than what would be observed with one compound or therapy delivered in the absence of the other. The effect of the two compounds or therapies can be partially additive, wholly additive, or great than additive (e.g., synergistic). The delivery can be such that the first compound or therapy delivered is still detectable when the second is delivered.

In some embodiments, the first compound or therapy and second compound or therapy can be administered simultaneously (e.g., at the same time), in the same or in separate compositions, or sequentially. Sequential administration refers to administration of one compound or therapy before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, compound or therapy. The order of administration of the first and secondary compound or therapy can also be reversed.

The combinations described herein can be a first line treatment for abnormal cell growth, e.g., cancer, i.e., it is used in a subject who has not been previously administered another drug intended to treat the cancer; a second line treatment for the cancer, i.e., it is used in a subject in need thereof who has been previously administered another drug intended to treat the cancer; a third or fourth treatment for the cancer, i.e., it is used in a subject who has been previously administered two or three other drugs intended to treat the cancer.

### Administration and Dosage

The combinations of this invention may be administered orally, parenterally, topically, rectally, or via an implanted reservoir, preferably by oral administration or administration by injection. In some cases, the pH of the composition (e.g., pharmaceutical composition) may be adjusted with pharmaceutically acceptable acids, bases or buffers to enhance the stability or efficacy of the composition.

In some embodiments, the subject is administered the composition (e.g., pharmaceutical composition) orally. In some embodiments the composition (e.g., pharmaceutical composition) is be orally administered in any orally acceptable dosage form including, but not limited to, liqui-gel tablets or capsules, syrups, emulsions and aqueous suspensions. Liqui-gels may include gelatins, plasticisers, and/or opacifiers, as needed to achieve a suitable consistency and may be coated with enteric coatings that are approved for use, e.g., shellacs. Additional thickening agents, for example gums, e.g., xanthum gum, starches, e.g., corn starch, or glutens may be added to achieve a desired consistency of the composition (e.g., pharmaceutical composition) when used as an oral dosage. If desired, certain sweetening and/or flavoring and/or coloring agents may be added.

In some embodiments, the subject is administered the composition (e.g., pharmaceutical composition) in a form suitable for oral administration such as a tablet, capsule, pill, powder, sustained release formulations, solution, and suspension. The composition (e.g., pharmaceutical composition) may be in unit dosage forms suitable for single administration of precise dosages. Pharmaceutical compositions may comprise, in addition to a compound as described herein a pharmaceutically acceptable carrier, and may optionally further comprise one or more pharmaceutically acceptable excipients, such as, for example, stabilizers, diluents, binders, and lubricants. In addition, the tablet may include other medicinal or pharmaceutical agents, carriers, and or adjuvants. Exemplary pharmaceutical compositions include compressed tablets (e.g., directly compressed tablets).

Tablets are also provided comprising the active or therapeutic ingredient (e.g., compound as described herein). In addition to the active or therapeutic ingredients, tablets may contain a number of inert materials such as carriers. Pharmaceutically acceptable carriers can be sterile liquids, such as water and oils, including those of petroleum, animal, vegetable or synthetic origin, such as peanut oil, sesame oil and the like. Saline solutions and aqueous dextrose can also be employed as liquid earners. Oral dosage forms for use in accordance with the present invention thus may be formulated in conventional manner using one or more pharmaceutically acceptable carriers comprising excipients and auxiliaries, which facilitate processing of the active ingredients into preparations which, can be used pharmaceutically.

Excipients can impart good powder flow and compression characteristics to the material being compressed. Examples of excipients are described, for example, in the Handbook of Pharmaceutical Excipients (5^{th} edition), Edited by Raymond C Rowe, Paul J. Sheskey, and Sian C. Owen; Publisher: Pharmaceutical Press.

For oral administration, the active ingredients, e.g., the compound as described herein can be formulated readily by combining the active ingredients with pharmaceutically acceptable carriers well known in the art. Such carriers enable the active ingredients of the invention to be formulated as tablets, pills, capsules, liquids, gels, syrups, slurries, powders or granules, suspensions or solutions in water or non-aqueous media, and the like, for oral ingestion by a subject. Pharmacological preparations for oral use can be made using a solid excipient, optionally grinding the resulting mixture, and processing the mixture of granules, after adding suitable auxiliaries if desired, to obtain, for example, tablets. Suitable excipients such as diluents, binders or disintegrants may be desirable.

The dosage may vary depending upon the dosage form employed and the route of administration utilized. The exact formulation, route of administration and dosage can be chosen by the individual physician in view of the subject's condition. (See e.g., Fingl, et al., 1975, in “The Pharmacological Basis of Therapeutics”). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician. A course of therapy can comprise one or more separate administrations of a compound as described herein. A course of therapy can comprise one or more cycles of a compound as described herein.

In some embodiments, a cycle, as used herein in the context of a cycle of administration of a drug, refers to a period of time for which a drug is administered to a subject. For example, if a drug is administered for a cycle of 21 days, the periodic administration, e.g., daily or twice daily, is given for 21 days. A drug can be administered for more than one cycle. Rest periods may be interposed between cycles. A rest cycle may be 1, 2, 4, 6, 8, 10, 12, 16, 20, 24 hours, 1, 2, 3, 4, 5, 6, 7 days, or 1, 2, 3, 4 or more weeks in length.

Oral dosage forms may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### EXAMPLES

In order that the invention described herein may be more fully understood, the following examples are set forth. The examples described in this application are offered to illustrate the pharmaceutical compositions and methods provided herein and are not to be construed in any way as limiting their scope.

### Example 1. Synergy of a Dual RAF/MEK Inhibitor and Autophagy Pathway Inhibitors

### Materials and Methods

### Autophagic Flux

Human KRAS mutant pancreatic cancer cell lines stably expressing a tandem fluorescent GFP-LC3-RFP-LC3ΔG reporter were grown in 2D conditions. Briefly, cells were seeded in 96-well plates and after an overnight incubation (17-22 hours), cells were treated with 1 μM of the RAF/MEK clamp avutometinib (VS-6766) for 24 hours. Following treatment, cells were collected and changes in autophagic flux were measured by flow cytometry. Autophagic flux was quantified by calculating the ratio of RFP/GFP in cell lines tested.

### Synergy Analysis

Parental human KRAS mutant pancreatic cancer cell lines were grown in 2D conditions. Briefly, cells were seeded in 96-well plates and after an overnight incubation (17-22 hours), cells were treated with the RAF/MEK clamp avutometinib (VS-6766)+/− the autophagy pathway inhibitor chloroquine (CQ) or apilimod (AP) for 5 days. Following drug treatment, live cell counting was performed and plotted to quantify effects on proliferation. Bliss synergy analysis were performed to calculate synergy scores between avutometinib+chloroquine and avutometinib+apilimod.

### Results

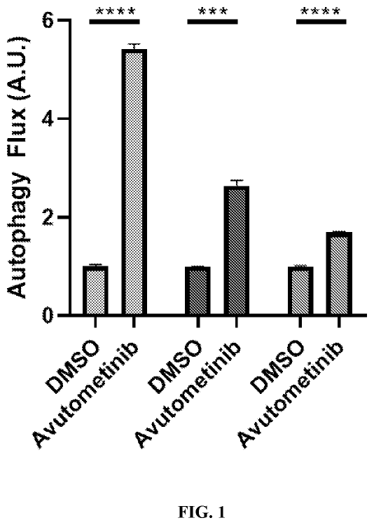

Autophagic flux was assessed in a panel of 3 human KRAS mutant pancreatic cancer cell lines stably expressing a tandem fluorescent GFP-LC3-RFP-LC3ΔG reporter (FIG. 1). Avutometinib treatment increased autophagic flux by two-to six-fold in all 3 human KRAS mutant pancreatic cancer cell lines tested. In FIG. 1, human KRAS mutant pancreatic cancer cell lines stably expressing a tandem fluorescent GFP-LC3-RFP-LC3ΔG reporter were treated with vehicle (DMSO) or avutometinib for 24 hours. Following treatment, cells were collected and changes in autophagic flux were measured by flow cytometry. Autophagic flux was quantified by calculating the ratio of RFP/GFP in cell lines tested.

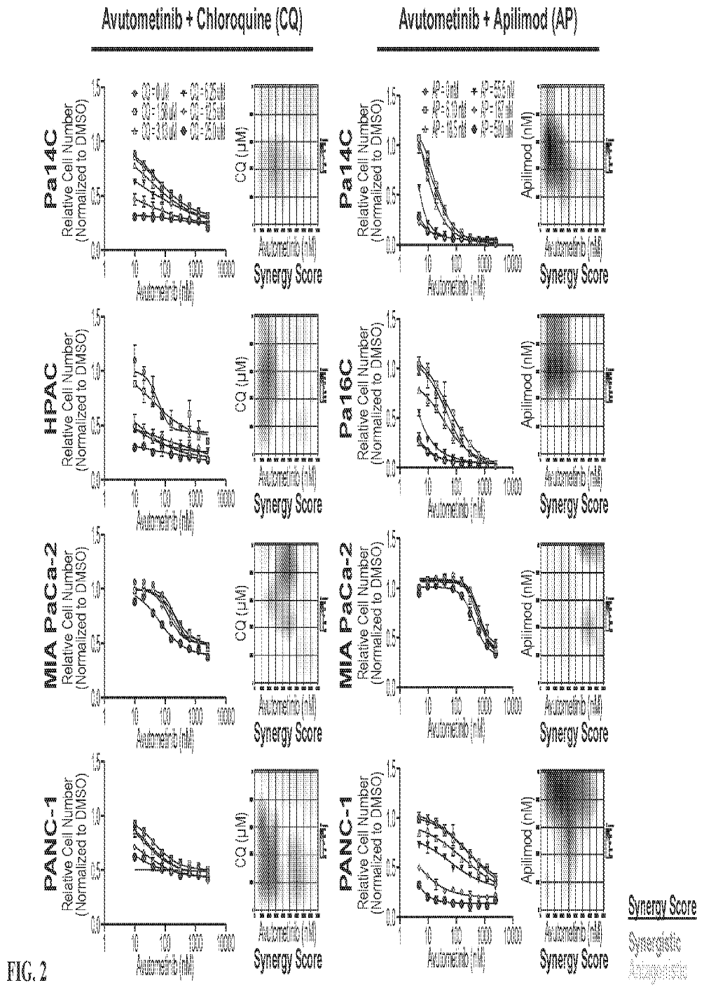

Proliferation assays were performed to determine whether chloroquine (CQ) or apilimod (AP) augmented the anti-proliferative activity of avutometinib in a panel of 4 human KRAS mutant pancreatic cancer cell lines (FIG. 2). Following drug treatment, live cell counting was performed and plotted to quantify effects on proliferation. Heatmaps indicating BLISS synergy scores are shown (Red=synergy, white=additivity, green=antagonism). As shown in FIG. 2, avutometinib was synergistic with chloroquine or apilimod in reducing cell viability of a panel of human KRAS mutant pancreatic cancer cell lines.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, some embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a cancer in a subject in need thereof, the method comprising administering to the subject an effective amount of a dual RAF/MEK inhibitor and an effective amount of an autophagy pathway inhibitor.

2. The method of claim 1, wherein the cancer is identified as having a RAS mutation.

3. The method of claim 1, wherein the cancer is identified as having a KRAS, NRAS, or HRAS mutation.

4. The method of claim 3, wherein the KRAS mutation is a mutation in KRAS G12V, KRAS G12D, KRAS G12A, KRAS G12R, KRAS G12S, or KRAS G12C.

5. The method of claim 3, wherein the KRAS mutation is a mutation in KRAS G13V, KRAS G13D, KRAS G13A, KRAS G13R, KRAS G13S, KRAS G13E, KRAS G12 dup, or KRAS G13C.

6. The method of claim 3, wherein the KRAS mutation is a mutation in KRAS Q61H, KRAS Q61K, KRAS Q61L, KRAS Q61R, KRAS Q61P, or KRAS Q61E.

7. The method of claim 1, wherein the cancer is identified as having RAF mutation.

8. The method of claim 1, wherein the cancer is identified as having a BRAF, ARAF, or CRAF mutation.

9. The method of claim 8, wherein the BRAF mutation is a BRAF V600 mutation.

10. The method of claim 1, wherein the cancer is identified as having MEK1 and/or MEK2 mutation.

11. The method of claim 1, wherein the cancer is identified as having NF1 alterations, KRAS amplification, NRAS amplification, and/or MYC amplification.

12. The method of claim 1, wherein the cancer is identified as having positive phospho-ERK protein expression (e.g., ≥10%, ≥20% or ≥30% of cells) detected by immunohistochemistry.

13. The method of any one of claims 1-12, wherein the dual RAF/MEK inhibitor is a compound of formula (I): 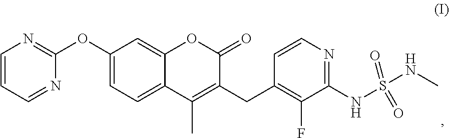 [SMILES: CNS(=O)(=O)Nc1nccc(Cc2c(C)c3ccc(Oc4ncccn4)cc3oc2=O)c1F]
or a pharmaceutically acceptable salt thereof.

14. The method of claim 13, wherein the dual RAF/MEK inhibitor is a compound of formula (I):

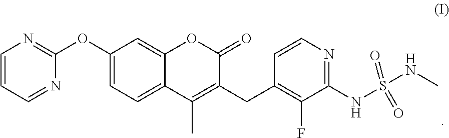 [SMILES: CNS(=O)(=O)Nc1nccc(Cc2c(C)c3ccc(Oc4ncccn4)cc3oc2=O)c1F]

15. The method of claim 13, wherein the dual RAF/MEK inhibitor is a potassium salt of the compound of formula (I).

16. The method of any one of claims 1-12, wherein the dual RAF/MEK inhibitor is IMM-1-104 or IK-595, or pharmaceutically acceptable salts thereof.

17. The method of any one of claims 1-12, wherein the dual RAF/MEK inhibitor is a compound of formula (II): 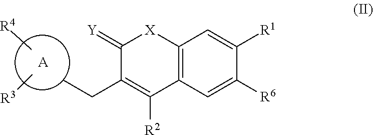
including pharmaceutically acceptable salts thereof, wherein:
Ring A is 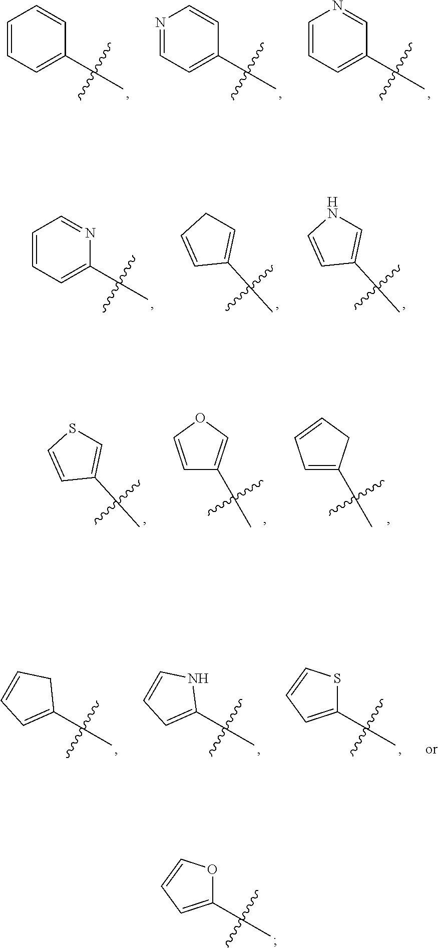
R¹, R², R³, and R⁴ are each independently selected from the group consisting of H, deuterium, hydroxyl, halogen, cyano, nitro, optionally substituted amino, optionally substituted C-amido, optionally substituted N-amido, optionally substituted ester, optionally substituted sulfonyl, optionally substituted S-sulfonamido, optionally substituted N-sulfonamido, optionally substituted sulfonate, optionally substituted O-thiocarbamyl, optionally substituted N-thiocarbamyl, optionally substituted N-carbamyl, optionally substituted O-carbamyl, optionally substituted urea, optionally substituted C1 to C6 alkoxy, optionally substituted C1 to C6 alkyl, optionally substituted C2 to C6 alkenyl, optionally substituted C2 to C6 alkynyl, optionally substituted C3 to C8 cycloalkyl, optionally substituted C6 to C10 aryl, optionally substituted C3 to C8 heterocyclyl, optionally substituted C3 to C10 heteroaryl, and L; R⁶ is selected from the group consisting of H, deuterium, hydroxyl, halogen, cyano, nitro, optionally substituted amino, optionally substituted C1 to C6 alkoxy, optionally substituted C1 to C6 alkyl, optionally substituted C2 to C6 alkenyl, and optionally substituted C2 to C6 alkynyl;
X is C(R⁵)₂, CH(R⁵), CH₂, —O—, 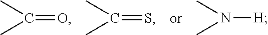
L is —Z₁—Z₂ or —Z₁—Z₂—Z₃;
Z₁, Z₂, and Z₃ are independently selected from the group consisting of —CH₂—, —O—, —S—, S═O, —SO₂—, C═O, —CO₂—, —NO₂, —NH—, —CH₂CCH, —CH₂CN, —NR⁵R^{5′}, —NH(CO)—, —(CO)NH—, —(CO)NR⁵R^{5′}—, —NH—SO₂—, —SO₂—NH—, —R⁵CH₂—, —R⁵O—, —R⁵S—, R⁵—S═O, —R⁵SO₂—, R⁵—C═O, —R⁵CO₂—, —R⁵NH—, —R⁵NH(CO)—, —R⁵(CO)NH—, —R⁵NH—SO₂—, —R⁵SO₂—NH—, —NHCH₂CO—, —CH₂R⁵—, —OR⁵—, —SR⁵—, S═O—R⁵, —SO₂R⁵—, C═O—R⁵, —CO₂R⁵—, —NHR⁵—, —NH(CO)R⁵—, —(CO)NHR⁵—, —NH—SO₂R⁵—, —SO₂—NHR⁵—, optionally substituted C1 to C6 alkyl, optionally substituted C3 to C8 cycloalkyl, optionally substituted C6 to C10 aryl, optionally substituted C3 to C8 heterocyclyl, optionally substituted C3 to C10 heteroaryl, —CH₂— (optionally substituted aryl), —CH₂— (optionally substituted C3 to C8 cycloalkyl), and —CH₂— (optionally substituted C3 to C10 heteroaryl); each R⁵ and R^{5′} are independently selected from H, deuterium, optionally substituted C1 to C6 alkyl, optionally substituted C2 to C6 alkenyl, optionally substituted C2 to C6 alkynyl, optionally substituted C3 to C8 carbocyclyl, optionally substituted C6 to C10 aryl, optionally substituted C3 to C8 heterocyclyl, and optionally substituted C3 to C10 heteroaryl; and
Y is CH₂, NH, or O, with the proviso that R¹ is not —O-pyrimidyl.

18. The method of any one of claims 1-12, wherein the dual RAF/MEK inhibitor is a compound selected from the compound of Table I, or a pharmaceutically acceptable salt thereof.

19. The method of any one of claims 1-18, wherein the dual RAF/MEK inhibitor is orally administered to the subject.

20. The method of any one of claims 1-19, wherein the dual RAF/MEK inhibitor is administered twice a week.

21. The method of any one of claims 1-20, wherein the dual RAF/MEK inhibitor is administered at a dose of 0.5 mg to about 10 mg per administration.

22. The method of claim 21, wherein the dual RAF/MEK inhibitor is dosed at 2.4 mg per administration.

23. The method of claim 21, wherein the dual RAF/MEK inhibitor is dosed at 3.2 mg per administration.

24. The method of claim 21, wherein the dual RAF/MEK inhibitor is dosed at 4 mg per administration.

25. The method of any one of claims 1-24, wherein the dual RAF/MEK inhibitor is dosed as a cycle comprising administering the dual RAF/MEK inhibitor for three weeks and then not administering the dual RAF/MEK inhibitor for one week.

26. The method of any one of claims 1-25, wherein the autophagy pathway inhibitor is AMPK inhibitor, L-type Ca2+ channel activator, USP10 and USP13 inhibitor, VPS34 inhibitor, or ULK inhibitor.

27. The method of any one of claims 1-25, wherein the autophagy pathway inhibitor is chloroquine, hydroxychloroquine, apilimod, 3-methyladenine, Wortmannin, LY294002, cycloheximide, bafilomycin A1, Lys05, leupeptin, E64d, Pepstatin A, (±)-Bay K 8644, Spautin 1, MRT 67307, MRT 68921, dorsomorphin, rapalogs, NSC-185058, DQ661, DCC-3116, PHY34, ENV-201, ERAS-5, LCC03 (5-(2′,4′-Difluorophenyl)-salicylanilide derivative), PTU (1-phenyl 2-thiourea), siroquine (JP001), SB02024, GCT-VPS34, Vescor01, S-130, SBI-0206965, SMER-28, or FMK-9A.

28. The method of any one of claims 1-27, wherein the autophagy pathway inhibitor is orally administered to the subject.

29. The method of any one of claims 1-28, wherein the autophagy pathway inhibitor is administered once daily.

30. The method of any one of claims 1-28, wherein the autophagy pathway inhibitor is administered twice daily.

31. The method of any one of claims 1-30, wherein the autophagy pathway inhibitor is dosed at 10 mg to 2000 mg per administration.

32. The method of any one of claims 1-31, wherein the autophagy pathway inhibitor is dosed at 100 mg to 1000 mg per administration.

33. The method of any one of claims 1-32, wherein the cancer is pancreatic cancer, gynecologic cancer (e.g., cervical cancer, ovarian cancer, uterine cancer, vaginal cancer, endometrial cancer, or vulvar cancer), liver cancer, prostate cancer, mesothelioma, breast cancer, bladder cancer, melanoma, lung cancer, colorectal cancer, thyroid cancer, glioblastoma, or renal cancer.

34. The method of any one of claims 1-32, wherein the cancer is melanoma, lung cancer, colorectal cancer, ovarian cancer, thyroid cancer, glioblastoma, or renal cancer.

35. The method of claim 33 or 34, wherein the lung cancer is non-small cell lung cancer.

36. The method of claim 33 or 34, wherein the lung cancer is metastatic non-small cell lung cancer.

37. The method of claim 33 or 34, wherein the melanoma is unresectable melanoma.

38. The method of claim 33 or 34, wherein the melanoma is metastatic melanoma.

39. The method of claim 33 or 34, wherein the cancer is colorectal cancer.

40. The method of claim 33 or 34, wherein the cancer is ovarian cancer.

41. The method of any one of claims 1-40, wherein the method inhibits autophagy initiation, nucleation, maturation, or degradation.

42. The method of any one of claims 1-40, wherein the method inhibits ULK1, ULK2, ATG13, FIP200, ATG101, VPS34, VPS15, ATG14L, Beclin-1, ATG4B, or lysosome function.

43. The method of any one of claims 1-42, further comprising administering to the subject an effective amount of a FAK inhibitor.

44. The method of claim 43, wherein the FAK inhibitor is defactinib, or a pharmaceutically acceptable salt thereof.

45. The method of claim 43 or 44, wherein the FAK inhibitor is dosed at about 100 mg to about 1000 mg.

46. The method of claim 45, wherein the FAK inhibitor is dosed at about 100 mg to about 400 mg per administration.

47. The method of claim 46, wherein the FAK inhibitor is dosed at 200 mg per administration.

48. The method of claim 46, wherein the FAK inhibitor is dosed at 400 mg per administration.

49. The method of any one of claims 43-48, wherein the FAK inhibitor is administered once daily.

50. The method of any one of claims 43-48, wherein the FAK inhibitor is administered twice daily.

51. The method of any one of claims 43-50, wherein the FAK inhibitor is dosed as a cycle, comprising administering the FAK inhibitor for three weeks and then not administering the FAK inhibitor for one week.

52. The method of any one of claims 43-51, wherein the FAK inhibitor is orally administered to the subject.
